(19) United States
(12) Patent Application Publication
(10) Pub. No.: US 2025/0222270 A1
(43) Pub. Date: Jul. 10, 2025

(54) PHOTOTHERAPY DEVICE

(71) Applicant: TERUMO KABUSHIKI KAISHA, Tokyo (JP)

(72) Inventors: Daiki TOHMA, Ichinomiya-shi (JP); Satoru SUEHARA, Kanagawa (JP); Mayu HATA, Kanagawa (JP)

(73) Assignee: TERUMO KABUSHIKI KAISHA, Tokyo (JP)

(21) Appl. No.: 19/093,627

(22) Filed: Mar. 28, 2025

Related U.S. Application Data
(63) Continuation of application No. PCT/JP2023/035015, filed on Sep. 26, 2023.

(30) Foreign Application Priority Data
Sep. 30, 2022 (JP) 2022158998
Sep. 30, 2022 (JP) 2022159001
Sep. 30, 2022 (JP) 2022159003

(51) Int. Cl. A61N 5/06

(52) U.S. Cl. CPC A61N 5/0603; A61N 2005/0611; A61N 2005/0632; A61N 2005/0664

(57) ABSTRACT

A phototherapy device includes an enclosure configured to enclose an internal region where a light emitter is disposed and define a longitudinal direction and an axial line extending in the longitudinal direction. The enclosure includes a light-blocking body that blocks at least a part of light emitted from the light emitter from being transmitted toward an outside of the internal region, and a window that transmits the light toward an outside of the internal region due to absence of the light-blocking body. The window extends from one end portion of the window located at one end portion of the enclosure in the longitudinal direction toward an other end portion of the enclosure. An other end portion of the window in the longitudinal direction is disposed over a limited angular range not extending over an entire periphery in a circumferential direction of the axial line.

### CROSS-REFERENCES TO RELATED APPLICATIONS

This application is a continuation of International Application No. PCT/JP2023/035015 filed on Sep. 26, 2023, which claims priority to Japanese Application No. 2022-158998 filed on Sep. 30, 2022, Japanese Application No. 2022-159001 filed on Sep. 30, 2022, and Japanese Application No. 2022-159003 filed on Sep. 30, 2022, the entire content of all four of which is incorporated herein by reference.

### TECHNOLOGICAL FIELD

The present disclosure relates to a phototherapy device.

### BACKGROUND DISCUSSION

A phototherapy device used for irradiating abnormal tissue with therapeutic light is known (see, for example, Japanese Patent No. 4122323 B1).

### SUMMARY

It is desirable that the phototherapy device does not irradiate normal tissues with light as much as possible.

A phototherapy device is disclosed, which is capable of irradiating the vicinity of the uterine orifice with light and at the same time irradiating the vaginal wall with light in a limited manner, for example, in the case of treating gynecological cancer.

One aspect of the present disclosure is as follows.
[1] A phototherapy device including: an enclosure that encloses an internal region where a light emitter is disposed and defines a longitudinal direction and an axial line extending in the longitudinal direction, in which the enclosure includes a light-blocking body that blocks at least a part of light emitted from the light emitter from being transmitted toward an outside of the internal region, and a window that transmits the light toward an outside of the internal region due to absence of the light-blocking body, the window extends from one end portion of the window located at one end portion of the enclosure in the longitudinal direction toward an other end portion of the enclosure, and an other end portion of the window in the longitudinal direction is disposed over a limited angular range not extending over an entire periphery in a circumferential direction of the axial line.
[2] The phototherapy device according to [1], in which a tip end of the one end portion of the window is located at a tip end of the one end portion of the enclosure.
[3] The phototherapy device according to [1] or [2], including: a lumen serving as a movement path of the light emitter.
[4] The phototherapy device according to [3], in which the light emitter is introduced into the internal region through the other end portion of the enclosure in the lumen serving as a movement path of the light emitter.
[5] The phototherapy device according to any one of [1] to [4], in which the other end portion of the window is defined by a straight line perpendicular to the longitudinal direction.
[6] The phototherapy device according to any one of [1] to [5], in which the enclosure includes an absorber that at least partially absorbs the light emitted from the light emitter, transmitted through the enclosure, and then incident on the enclosure from outside.
[7] The phototherapy device according to any one of [1] to [6], in which the other end portion of the enclosure has a labeling body indicating an orientation of the window in the circumferential direction.
[8] The phototherapy device according to any one of [1] to [7], in which the enclosure is a balloon configured to be deformable from a contracted mode to an expanded mode.
[9] The phototherapy device according to [8], including: a lumen serving as a movement path of fluid that deforms the enclosure from the contracted mode to the expanded mode.
[10] The phototherapy device according to [9], in which the fluid is introduced into the internal region through the other end portion of the enclosure in the lumen serving as a movement path of the fluid.
[11] A phototherapy method using the phototherapy device according to [1], the method including: inserting the enclosure into a living body; rotating the enclosure in the circumferential direction in accordance with a position of a tumor; emitting the light from the enclosure through the window; and removing the enclosure from the living body.
[12] The phototherapy method according to [11], including: expanding a balloon as the enclosure.
[13] The phototherapy method according to [12], including: contracting the balloon, in which the contracting is performed after the emitting and before the removing of the enclosure.
[14] The phototherapy method according to [11], including: visually recognizing a uterine orifice and an affected area with a colposcope or another visual recognition instrument inserted into a vagina; and removing the visual recognition instrument from an inside of the vagina, in which the visual recognizing is performed before the inserting, the rotating, the emitting, the removing, and the expanding, and the removing of the visual recognition instrument is performed after the inserting and before the emitting.
[15] The phototherapy method according to [11], including: confirming that the enclosure has been abutted against a deepest part of a vagina by the inserting.
[16] The phototherapy method according to [12], in which the inserting, the expanding, the rotating, the emitting, and the removing are performed in this order.
[17] The phototherapy method according to [12], in which the inserting, the rotating, the expanding, the emitting, and the removing are performed in this order.
[18] The phototherapy method according to [12], in which the rotating, the inserting, the expanding, the emitting, and the removing are performed in this order.
[19] The phototherapy method according to [12], in which the expanding, the inserting, the rotating, the emitting, and the removing are performed in this order.
[20] The phototherapy method according to [12], in which the expanding, the rotating, the inserting, the emitting, and the removing are performed in this order.
[21] The phototherapy method according to [12], including: removing a protective sheath from inside the living body, in which the inserting is performed in a state where the balloon in a contracted mode is protected by the protective sheath, the removing of the protective sheath is performed after the inserting, and the expanding is performed after the removing of the protective sheath.
[22] The phototherapy method according to [11], including: irradiating an entire (or whole) inside of the living body with the light having output power smaller than output power in the emitting, in which the irradiating is performed after the emitting.
[23] The phototherapy method according to [12], including: confirming that the balloon has been expanded by using ultrasonic waves, in which the expanding is performed after the inserting, and the confirming is performed after the expanding.
[24] The phototherapy method according to [11], including: re-emitting the light by changing a position of an emission range in the living body after the emitting.
[25] The phototherapy method according to [24], including: changing a position of the enclosure by rotating the enclosure in the circumferential direction or moving the enclosure in the longitudinal direction, in which the changing is performed after the emitting and before the re-emitting.
[26] The phototherapy method according to [25], including: repeating the changing and the re-emitting a plurality of times in this order after the emitting.
[27] The phototherapy method according to [11], in which a first phototherapy device is used as the phototherapy device, the first phototherapy device including a first enclosure that encloses a first internal region in which a first light emitter is disposed and defines a first longitudinal direction and a first axial line extending in the first longitudinal direction, the first enclosure including a first light-blocking body that blocks at least part of first light emitted from the first light emitter from being transmitted toward an outside of the first internal region and a first window that transmits the first light toward an outside of the first internal region due to absence of the first light-blocking body, the first window extending from one end portion of the first window located at one end portion of the first enclosure in the first longitudinal direction toward the other end portion of the first enclosure, and the other end portion of the first window in the first longitudinal direction being disposed over a limited angular range not extending over an entire periphery in a circumferential direction of the first axial line, a second phototherapy device is used, the second phototherapy device including a second enclosure that encloses a second internal region in which a second light emitter is disposed and defines a second longitudinal direction and a second axial line extending in the second longitudinal direction, the second enclosure including a second light-blocking body that blocks at least part of second light emitted from the second light emitter from being transmitted toward an outside of the second internal region, and a second window that transmits the second light toward an outside of the second internal region due to absence of the second light-blocking body, and the second window being disposed in a portion other than both end portions of the second enclosure in the second longitudinal direction, and the phototherapy method comprises: inserting, as the inserting using the first phototherapy device, the first enclosure into the living body; rotating, as the rotating using the first phototherapy device, the first enclosure in a first circumferential direction in accordance with a position of the tumor; emitting, as the emitting using the first phototherapy device, the first light from the first enclosure through the first window; removing, as the removing using the first phototherapy device, the first enclosure from the living body; inserting, using the second phototherapy device, the second enclosure into the living body; rotating, using the second phototherapy device, the second enclosure in the second circumferential direction in accordance with a position of the tumor; emitting, using the second phototherapy device, the second light from the second enclosure through the second window; and removing, using the second phototherapy device, the second enclosure from the living body.
[28] The phototherapy method according to [27], in which the inserting using the second phototherapy device, the rotating using the second phototherapy device, the emitting using the second phototherapy device, and the removing using the second phototherapy device are performed after the inserting using the first phototherapy device, the rotating using the first phototherapy device, the emitting using the first phototherapy device, and the removing using the first phototherapy device.
[29] The phototherapy device according to [1], including: a lumen serving as a movement path of fluid that deforms the enclosure from a contracted mode to an expanded mode, in which the light emitter is disposed on the axial line in the internal region, and the light-blocking body is disposed at least at one end portion of the enclosure in the longitudinal direction so as to have a different thickness depending on a position.
[30] The phototherapy device according to [29], in which the fluid is introduced into the internal region through the one end portion of the balloon in the lumen serving as a movement path of the fluid.
[31] The phototherapy device according to [29], in which a thickness of the light-blocking body is set such that a thickness of the light-blocking body at a predetermined position on a peripheral wall is smaller than a thickness of the light-blocking body at a predetermined position on an end wall in a proximal end portion of the balloon.
[32] The phototherapy device according to [29], in which a thickness of the light-blocking body is set such that a thickness of the light-blocking body at a predetermined position of an end wall is smaller than a thickness of the light-blocking body on a radially inner side of the predetermined position of the end wall in a proximal end portion of the balloon.
[33] The phototherapy device according to [29], in which in a case where the light-blocking body is disposed on a peripheral wall of the balloon, a thickness of the light-blocking body is set such that a thickness of the light-blocking body at a first predetermined position on the peripheral wall is smaller than a thickness of the light-blocking body at a second predetermined position on the peripheral wall proximal of the first predetermined position.
[34] The phototherapy device according to [29], in which in a case where the window is disposed to be biased to one side as viewed in the longitudinal direction, a thickness of the light-blocking body is set such that a thickness of the light-blocking body at a first predetermined position is larger than a thickness at a second predetermined position shifted from the first predetermined position toward the window in a circumferential direction.
[35] The phototherapy device according to [29], in which a thickness of the light-blocking body is set such that a thickness at an edge portion of the light-blocking body forming a boundary with the window is smaller than a thickness of the light-blocking body at other positions.
[36] The phototherapy device according to [29], in which a thickness of the light-blocking body is set to gradually change as the position changes.
[37] A method for manufacturing the phototherapy device according to any one of [29] to [36], in which the light-blocking body is formed by a dipping method.
[38] The phototherapy device according to [1], in which the light emitter is disposed on an axial center of the enclosure, and the light emitter is disposed so as not to overlap the window as viewed in a radial direction.
[39] The phototherapy device according to [38], in which the one end portion of the window is located at the one end portion of the hollow body.
[40] The phototherapy device according to [38], including: a lumen serving as a movement path of the light emitter.
[41] The phototherapy device according to [40], in which the light emitter is introduced into the internal region through the other end portion of the hollow body in the lumen serving as a movement path of the light emitter.
[42] The phototherapy device according to [40], including: a tube that forms the lumen serving as a movement path of the light emitter, in which the tube has a marker indicating a position where the light emitter is to be disposed.
[43] The phototherapy device according to [40], including: a stopper that restricts movement of the light emitter in an introduction direction in the lumen serving as a movement path of the light emitter, at a position where the light emitter is to be disposed.
[44] The phototherapy device according to [38], in which a tip end of the one end portion of the window is located at a tip end of the one end portion of the hollow body.
[45] The phototherapy device according to [38], in which the hollow body is a balloon configured to be deformable from a contracted mode to a developed mode.
[46] The phototherapy device according to [45], including: a lumen serving as a movement path of fluid that deforms the hollow body from the contracted mode to the developed mode.
[47] The phototherapy device according to [46], in which the fluid is introduced into the internal region through the other end portion of the hollow body in the lumen serving as a movement path of the fluid.
[48] A phototherapy device including: a balloon that forms an internal space in which a light emitter is disposed and defines a longitudinal direction and an axial line extending in the longitudinal direction; and a lumen serving as a movement path of fluid that deforms the balloon from a contracted mode to an expanded mode, in which the light emitter is disposed on the axial line in the internal space, the balloon includes a light-blocking body that blocks at least a part of light emitted from the light emitter from being transmitted toward an outside of the internal space, and a window that transmits the light toward an outside of the internal space due to absence of the light-blocking body, and the light-blocking body is disposed at least at one end portion of the balloon in the longitudinal direction so as to have a different thickness depending on a position.
[49] The phototherapy method according to any one of [11] to [28], in which the living body is a hollow organ.
[50] The phototherapy method according to any one of [11] to [28], and [49], including: administering a substance containing a photosensitizer before the inserting of the enclosure into the living body.
[51] The phototherapy method according to [50], in which the photosensitizer is a substance containing a phthalocyanine dye.
[52] The phototherapy method according to any one of [11] to [28] and [49] to [51], in which the substance containing the photosensitizer is an antibody-photosensitizer, and the method comprises administering the antibody-photosensitizer before the inserting of the enclosure into the living body.
[53] The phototherapy device according to any one of [1] to [10] and [29] to [48], in which a wavelength of the light is in a range of 660 nm to 740 nm.

According to the present disclosure, for example, in the case of treating gynecological cancer, it is possible to provide a phototherapy device capable of irradiating the vicinity of the uterine orifice with light and at the same time irradiating the vaginal wall with light in a limited manner.

### BRIEF DESCRIPTION OF THE DRAWINGS

FIG. 1 is a perspective view in an expanded mode of a phototherapy device according to a first embodiment (first aspect).

FIG. 2 is a plan view of the phototherapy device illustrated in FIG. 1 as viewed from a radial direction.

FIG. 3 is a plan view of the phototherapy device illustrated in FIG. 1 as viewed from a longitudinal direction.

FIG. 4 is a cross-sectional view taken along line AA-AA of FIG. 3.

FIG. 5 is a plan view of the phototherapy device illustrated in FIG. 1 as viewed from a radial direction in the contracted mode.

FIG. 6 is a plan view of the phototherapy device illustrated in FIG. 1 as viewed from a longitudinal direction in the contracted mode.

FIG. 7 is a perspective view of a phototherapy device according to a second embodiment (first aspect).

FIG. 8 is a plan view of the phototherapy device illustrated in FIG. 7 as viewed from a radial direction.

FIG. 9 is a plan view seen from an angle different by 90° in the circumferential direction from FIG. 8.

FIG. 10 is a perspective view of a phototherapy device according to a third embodiment (first aspect).

FIG. 11 is a plan view of the phototherapy device illustrated in FIG. 10 as viewed from a radial direction.

FIG. 12 is a perspective view of a phototherapy device used in combination with the phototherapy device illustrated in FIG. 10.

FIG. 13 is a perspective view of a phototherapy device according to a fourth embodiment (first aspect).

FIG. 14 is a plan view of the phototherapy device illustrated in FIG. 13 as viewed from a radial direction.

FIG. 15 is a perspective view of a phototherapy device according to a fifth embodiment (first aspect).

FIG. 16 is a perspective view of a phototherapy device according to a sixth embodiment (first aspect).

FIG. 17 is an explanatory view for explaining a phototherapy method according to an embodiment (first aspect).

FIG. 18 illustrates a state where an enclosure is inserted into a living body from the state illustrated in FIG. 17.

FIG. 19 illustrates a state where the enclosure is expanded from the state illustrated in FIG. 18.

FIG. 20 is a flowchart illustrating a procedure of the phototherapy method described in FIG. 17.

FIG. 21 is a flowchart illustrating a procedure of a re-emission step in the phototherapy method described in FIG. 17.

FIG. 22 is a perspective view in an expanded mode of a phototherapy device according to an embodiment (second aspect).

FIG. 23 is a plan view of the phototherapy device illustrated in FIG. 22 as viewed from a radial direction.

FIG. 24 is a plan view of the phototherapy device illustrated in FIG. 22 as viewed from a longitudinal direction.

FIG. 25 is a cross-sectional view taken along line BA-BA of FIG. 24.

FIG. 26 is a plan view of the phototherapy device illustrated in FIG. 22 as viewed from the radial direction in the contracted mode.

FIG. 27 is a plan view of the phototherapy device illustrated in FIG. 22 as viewed from the longitudinal direction in the contracted mode.

FIG. 28 is a cross-sectional view schematically illustrating a modification of a window.

FIG. 29 is a cross-sectional view schematically illustrating another modification of the window.

FIG. 30 is an explanatory view for explaining setting of the thickness of a reflector.

FIG. 31 is a perspective view in a developed mode of the phototherapy device according to the first embodiment (third aspect).

FIG. 32 is a plan view of the phototherapy device illustrated in FIG. 31 as viewed from a radial direction.

FIG. 33 is a plan view of the phototherapy device illustrated in FIG. 31 as viewed from a longitudinal direction.

FIG. 34 is a cross-sectional view taken along line CA-CA of FIG. 33.

FIG. 35 is a plan view of the phototherapy device illustrated in FIG. 31 as viewed from a radial direction in the contracted mode.

FIG. 36 is a plan view of the phototherapy device illustrated in FIG. 31 as viewed from a longitudinal direction in the contracted mode.

FIG. 37 is a cross-sectional view illustrating an example of a stopper.

FIG. 38 is a cross-sectional view illustrating an example of the stopper.

FIG. 39 is a perspective view of a phototherapy device according to a second embodiment (third aspect).

FIG. 40 is a plan view of the phototherapy device illustrated in FIG. 39 as viewed from a radial direction.

FIG. 41 is a plan view seen from an angle different by 90° in the circumferential direction from FIG. 40.

### DETAILED DESCRIPTION

Set forth below with reference to the accompanying drawings is a detailed description of embodiments of a phototherapy device. Hereinafter, embodiments of a first aspect, a second aspect, and a third aspect of the present disclosure will be sequentially illustrated and described in detail with reference to the drawings.

First, an embodiment of the first aspect of the present disclosure will be described.

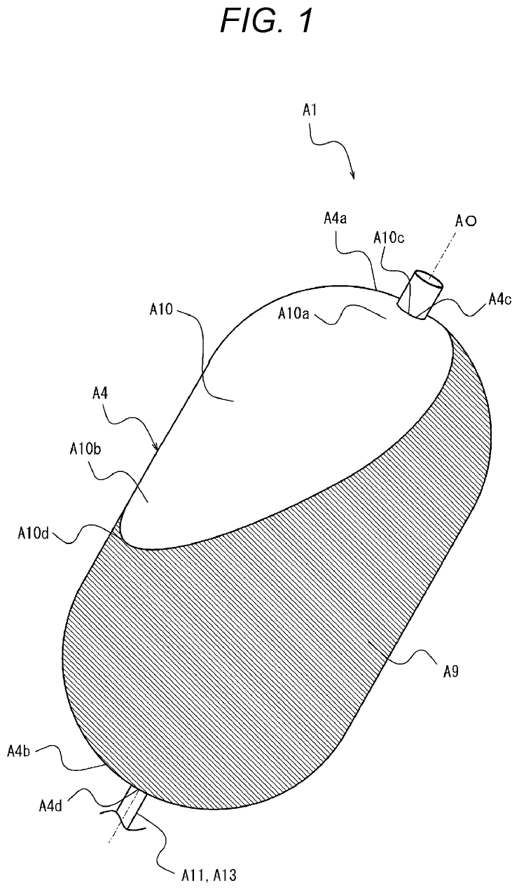

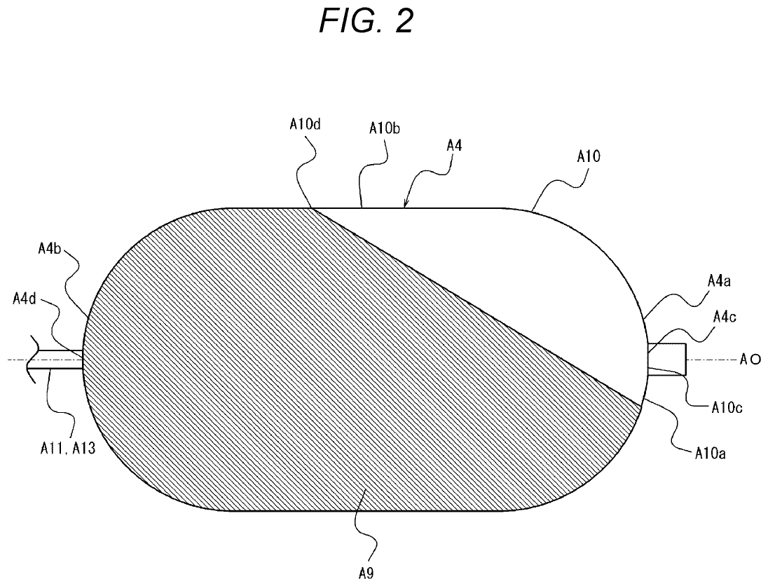

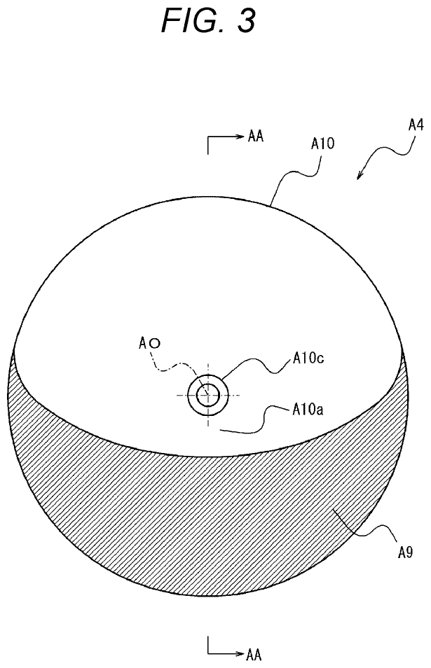

A phototherapy device A1 according to a first embodiment of the first aspect of the present disclosure illustrated in FIGS. 1 to 6 is used to irradiate abnormal tissue with therapeutic light in a phototherapeutic treatment such as photoimmunotherapy (PIT) or photodynamic therapy (PDT). The photoimmunotherapy is a therapeutic method disclosed in Japanese Patent No. 6127045 in which a drug adsorbed to a target cell is irradiated with light to destroy the target cell. The target cell is a tumor cell such as a cancer cell or a cell of a precancerous lesion. In this therapeutic method, a photosensitizer in which an antibody that specifically binds to only a specific antigen on the surface of a tumor cell and a photosensitizer paired with the antibody are adsorbed is used as a drug. The antibody is not particularly limited, and examples of the antibody can include panitumumab, trastuzumab, HuJ591, pertuzumab, lapatinib, palbociclib, olaparib, and the like. The photosensitizer can be, for example, but not limited to, hydrophilic phthalocyanine, which is a substance reactive to near infrared rays having a wavelength of about 700 nm (IR700). When IR700 receives near infrared rays having a wavelength of about in a range of 660 nm to 740 nm, a ligand of a functional group that ensures water solubility is broken, and a structural change from water solubility to hydrophobicity occurs. The membrane protein is extracted by this structural change, a hole is formed in the cell membrane, and water enters the cell, whereby the tumor cell can be ruptured and destroyed. In addition, the IR700 is excited by receiving near infrared rays and emits fluorescence having a wavelength different from the excitation wavelength. For example, when the IR700 is excited by receiving near infrared rays having a wavelength around 690 nm, the IR700 emits fluorescence having a wavelength around 700 nm. The IR700 undergoes a structural change while emitting fluorescence by photoreaction, and does not emit fluorescence when it destroys tumor cells and serves as a drug. The light can be, for example, laser light. Here, the wavelength of light can be, for example, in a range of 660 nm to 740 nm, which is a wavelength that activates the IR700 to be described later.

The phototherapy device A1 includes a light emitting device A2 having a light emitter A2a, an enclosure A4 enclosing an internal region A3 in which the light emitter A2a is disposed and defining a longitudinal direction and an axial line AO extending in the longitudinal direction, and a lumen A5 (hereinafter, also referred to as an optical lumen A5) serving as a movement path of the light emitter A2a. Note that a direction along a straight line perpendicular to the axial line AO is also referred to as a radial direction or simply a radial direction of the axial line AO, and a direction around the axial line AO is also referred to as a circumferential direction or simply a circumferential direction of the axial line AO. In the present embodiment, the axial line AO is the central axial line of the enclosure A4. The axial line AO is not limited to the central axial line.

The enclosure A4 is elongated in a predetermined direction, and defines a longitudinal direction as the predetermined direction. Further, the enclosure A4 has a tubular shape in which both end portions in the longitudinal direction are closed around the axial line AO. The light emitter A2a is disposed at a predetermined position on the axial line AO in the internal region A3. The arrangement of the light emitter A2a in the longitudinal direction in the internal region A3 can be appropriately set.

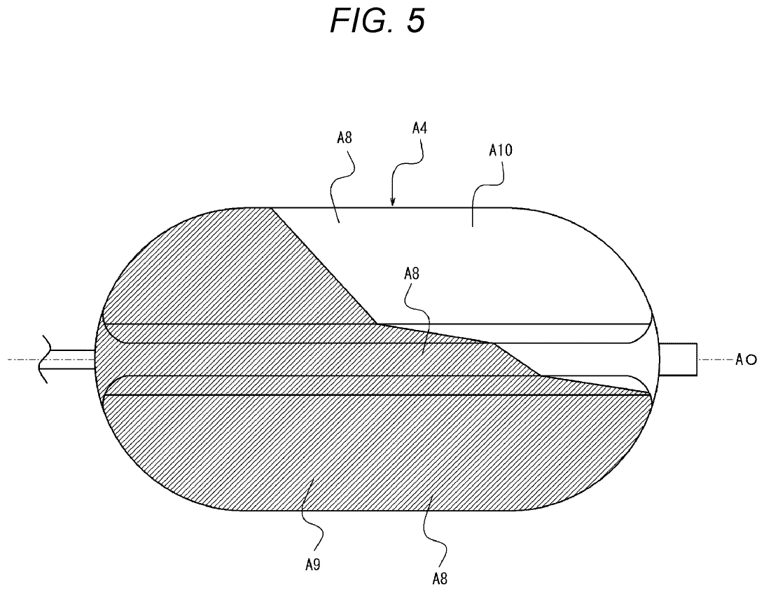

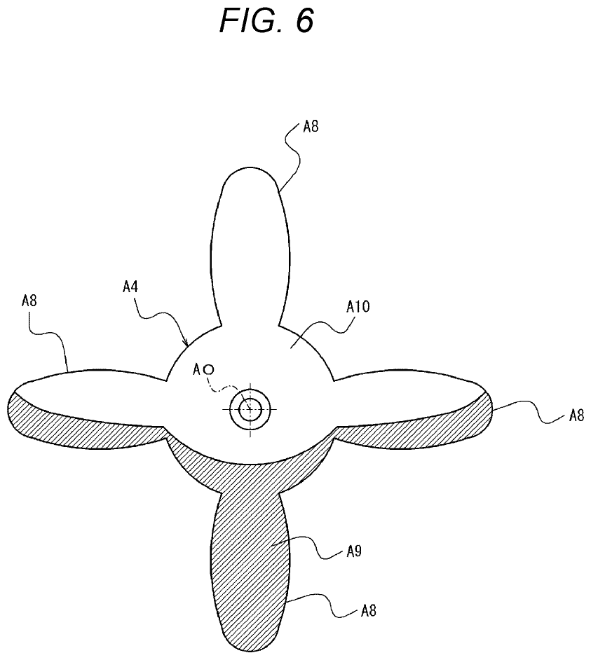

The enclosure A4 is a balloon that is configured to be deformable from the contracted mode illustrated in FIGS. 5 and 6 to the expanded mode illustrated in FIGS. 1 to 4, and the phototherapy device A1 further includes a lumen A6 (hereinafter, also referred to as a fluid lumen A6) serving as a movement path of fluid that deforms the balloon from the contracted mode to the expanded mode. The enclosure A4 can be deformed from the contracted mode to the expanded mode by the pressure of the fluid moving within the fluid lumen A6 and entering the internal region A3. The enclosure A4 (balloon) may be configured to be deformable from the contracted mode to the expanded mode by means other than fluid. The internal region A3 is an internal space that allows fluid to flow in when the balloon deforms from the contracted mode to the expanded mode.

In the expanded mode, the enclosure A4 has a cylindrical shape in which both end portions in the longitudinal direction are closed around the axial line AO. More specifically, the enclosure A4 includes a peripheral wall A7a having a cylindrical shape in the expanded mode, one end wall A7b having a convex curved shape continuous with one end portion of the peripheral wall A7a and protruding outward in the longitudinal direction in the expanded mode, and another end wall A7b having a convex curved shape continuous with the other end portion of the peripheral wall A7a and protruding outward in the longitudinal direction in the expanded mode.

Both or any one of the end walls A7b of the enclosure A4 may have a convex shape other than a convex curved shape in the expanded mode. The enclosure A4 may have a configuration in which the peripheral wall A7a has a tubular shape other than a cylindrical shape, such as an elliptical cylindrical shape in the expanded mode. That is, the enclosure A4 may have a tubular shape other than a cylindrical shape in which both end portions in the longitudinal direction are closed around the axial line AO in the expanded mode.

The contracted mode is a mode in which the enclosure A4 is folded into a predetermined shape so that the internal region A3 is reduced from the expanded mode. The predetermined shape may be a shape in which a plurality of pleats A8 are arranged in the circumferential direction, and may be, for example, a shape in which four pleats A8 are formed as illustrated in FIGS. 5 and 6.

The configuration of the enclosure A4 is not limited to the balloon that is configured to be deformable from the contracted mode to the expanded mode, and may be, for example, a configuration that always corresponds to the above-described expanded mode. In this case, the internal region A3 is not limited to a space, and may be configured as a region made of a translucent material such as resin, for example.

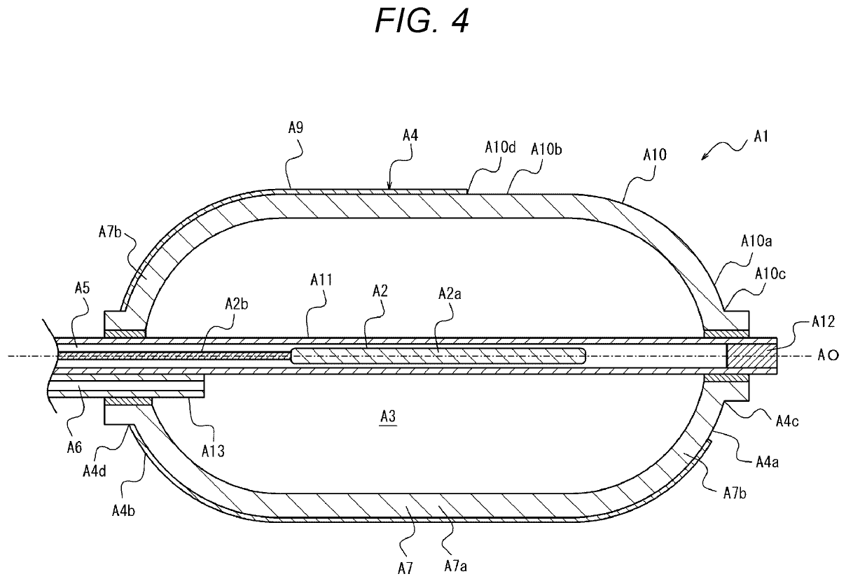

As illustrated in FIG. 4, the light emitting device A2 further includes a light source and an elongated light guide unit A2b that transmits the light emitted from the light source to the light emitter A2a. The light guide unit A2b is configured by, for example, a light guide such as an optical fiber, and is continuous with the light source at one end portion and is continuous with the light emitter A2a at the other end portion. In addition, the light guide unit A2b may have an optical connector that is connected to the light guide and is configured to be detachable from the light source.

The light emitter A2a extends in an elongated shape along the axial center, and is configured to be capable of emitting light transmitted from the light source via the light guide unit A2b in the radial direction of the axial center from a portion over a predetermined width in the direction along the axial center. More specifically, the light emitter A2a can emit light toward the entire region in the circumferential direction of the axial center.

The enclosure A4 includes a light-blocking body A9 that blocks at least part of light emitted from the light emitter A2a from being transmitted toward the outside of the internal region A3, and a window A10 that transmits light toward the outside of the internal region A3 due to absence of the light-blocking body A9. More specifically, the enclosure A4 includes a main body A7 having a peripheral wall A7a and two end walls A7b, and a light-blocking body A9 covering a part of an outer surface of the main body A7, and the window A10 is configured by a portion of the main body A7 that is not covered with the light-blocking body A9. The main body A7 is formed of a light transmitting material, and the light-blocking body A9 is formed of a light blocking material. The light-blocking body A9 may be configured as a reflector that at least partially reflects the light emitted from the light emitter A2a toward the internal region A3. In this case, the light-blocking body A9 is formed of a light reflective material. The main body A7 can be made of, for example, a resin material, and is preferably made of polyethylene terephthalate, polyurethane, nylon, or the like. The light-blocking body A9 can be formed of, for example, various metal materials (for example, titanium oxide, barium sulfate, zinc oxide, silver, aluminum, and the like). The thickness of the light-blocking body A9 is preferably in a range of 1 μm to 500 μm, more specifically in a range of 10 μm to 450 μm. According to the light-blocking body A9, the light emitted from the light emitter A2a can be collected and efficiently emitted to a limited range through the window A10.

The method for forming the light-blocking body A9 is not particularly limited, and the light-blocking body A9 can be formed by, for example, coating to the main body A7. The coating method is not particularly limited, and for example, a dipping method, a spray coating method, a roll coating method, a screen printing method, a vacuum vapor deposition method, a low vacuum sputtering method, or the like can be used. The light-blocking body A9 is not limited to cover a part of the outer surface of the main body A7, but may cover a part of the inner surface of the main body A7, for example.

The window A10 extends from one end portion (hereinafter, also referred to as a distal end portion A10a) of the window A10 located at one end portion (hereinafter, also referred to as a distal end portion A4a) of the enclosure A4 in the longitudinal direction toward the other end portion (hereinafter, also referred to as a proximal end portion A4b) of the enclosure A4. In addition, a tip end A10c of the distal end portion A10a of the window A10 is located at a tip end A4c of the distal end portion A4a of the enclosure A4. That is, the window A10 is disposed in a region including the center of the enclosure A4 as viewed in the longitudinal direction.

The other end portion (hereinafter, also referred to as a proximal end portion A10b) of the window A10 in the longitudinal direction is disposed over a limited angular range not extending over the entire periphery in the circumferential direction of the axial line AO. That is, the proximal end portion A10b of the window A10 is disposed only in a part in the circumferential direction. An angular range in which the window A10 is disposed is set so as to be gradually narrowed from the distal end portion A10a toward the proximal end portion A10b of the window A10. A tip end A10d of the proximal end portion A10b of the window A10 is located on the peripheral wall A7a. More specifically, the tip end A10d of the proximal end portion A10b of the window A10 is located on the proximal end side with respect to the center of the enclosure A4 in the longitudinal direction. The angular range in the circumferential direction of the axial line AO at the tip end A10d can be, for example, a range of 45 degrees to 180 degrees, more specifically a range of 60 degrees to 120 degrees, preferably 90 degrees.

The light emitter A2a is introduced into the internal region A3 through a tip end A4d of the proximal end portion A4b of the enclosure A4 in the optical lumen A5. The optical lumen A5 is formed by a tube A11 (hereinafter, also referred to as an optical tube A11) arranged coaxially with the axial line AO, wherein the optical tube A11 passes through a proximal end portion A4b and a distal end portion A4a of the enclosure A4. More specifically, the optical tube A11 is bonded to the end wall A7b at the proximal end portion A4b of the enclosure A4, and bonded to the end wall A7b at the distal end portion A4a of the enclosure A4. The optical tube A11 is closed by a closing member A12 at the distal end portion A4a of the enclosure A4. The optical tube A11 has optical transparency.

The optical tube A11 may have a marker such as a contrast marker having radiopacity serving as a mark for arranging the light emitter A2a at the predetermined position. Alternatively, the optical tube A11 may have a stopper that restricts the movement of the light emitter A2a in the introduction direction in the optical lumen A5 by hitting the light emitter A2a at the predetermined position. The stopper may be a diameter-reduced portion disposed in the optical tube A11, or may be a member disposed in the optical tube A11.

Fluid is introduced into the internal region A3 through the proximal end portion A4b of the enclosure A4 within the fluid lumen A6 to deform the enclosure A4 from a contracted mode to the expanded mode. As the fluid, for example, air, water, saline, or the like can be used. The fluid lumen A6 is formed by a tube A13 (hereinafter, also referred to as a fluid tube A13) disposed adjacent to and in parallel with the optical tube A11, and the fluid tube A13 passes through the proximal end portion A4b of the enclosure A4. More specifically, the fluid tube A13 is adhered to the end wall A7b at the proximal end portion A4b of the enclosure A4. The fluid tube A13 opens in the internal region A3.

The fluid lumen A6 is not limited to the configuration disposed in parallel with the optical lumen A5, and for example, may be disposed on the radial outside of the optical lumen A5 coaxially with the optical lumen A5, or may be configured with a common lumen that is configured to be used as both the fluid lumen A6 and the optical lumen A5.

According to the phototherapy device A1 of the present embodiment, for example, in the case of treating gynecological cancer, the enclosure A4 is deformed from the contracted mode to the expanded mode in front of the uterine orifice, and light can be widely emitted from the distal end portion A10a of the window A10 to the vicinity of the uterine orifice, and at the same time, light can be limitedly emitted to the vaginal wall from the remaining portion of the window A10 in the circumferential direction. Therefore, it is possible to treat the abnormal site while suppressing the irradiation of the normal site of the vaginal wall with light. According to such a phototherapy device A1, it is possible to safely treat a certain case of gynecological cancer. Note that the phototherapy device A1 may be applied to cancer other than gynecological cancer (for example, esophageal cancer or gastric cancer). For example, in a case where the phototherapy device A1 is applied to a hollow organ such as a digestive tract (esophagus, stomach, small intestine, large intestine, etc.), a urinary tract, and a blood vessel, the phototherapy device A1 may be used through a treatment tool channel of an endoscope device inserted into the hollow organ. Even in such a case, the normal tissue other than the abnormal site (lesion) can be prevented from being directly irradiated with light, and the abnormal site can be exclusively irradiated with light.

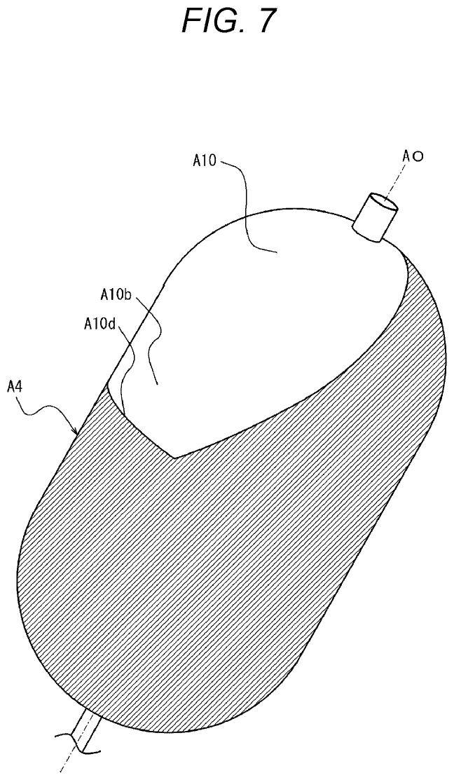

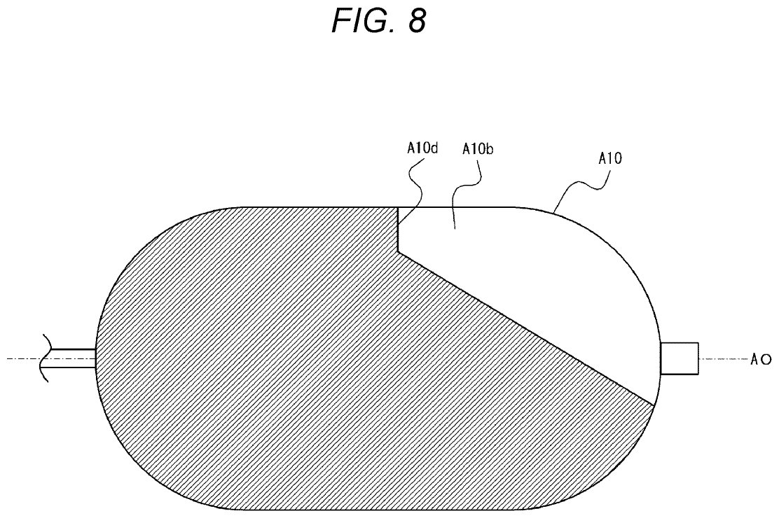

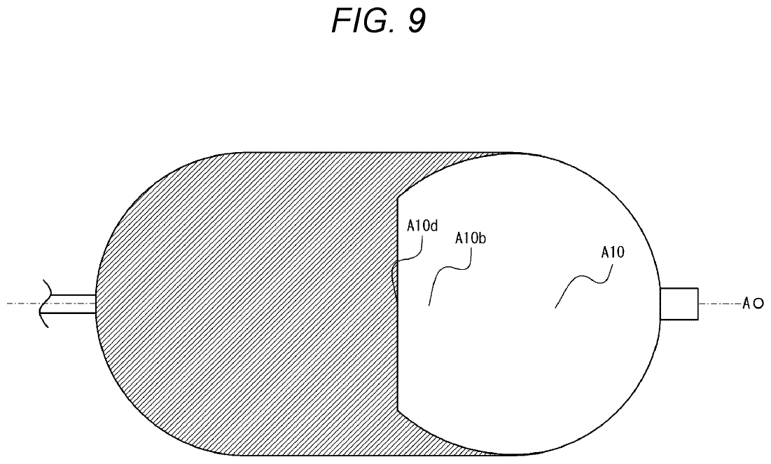

As in the second embodiment illustrated in FIGS. 7 to 9, the window A10 may have a configuration in which the proximal end portion A10b of the window A10 is defined by a straight line perpendicular to the longitudinal direction (a configuration in which the tip end A10d of the proximal end portion A10b of the window A10 extends along the circumferential direction). According to such a configuration, since light can be emitted with a constant width in the circumferential direction at the tip end A10d of the proximal end portion A10b of the window A10, efficient light irradiation can be performed in a certain case of gynecological cancer.

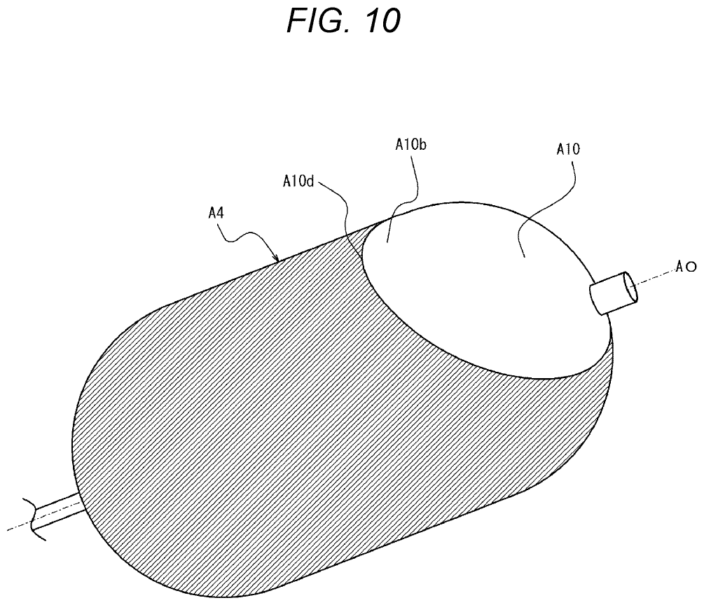

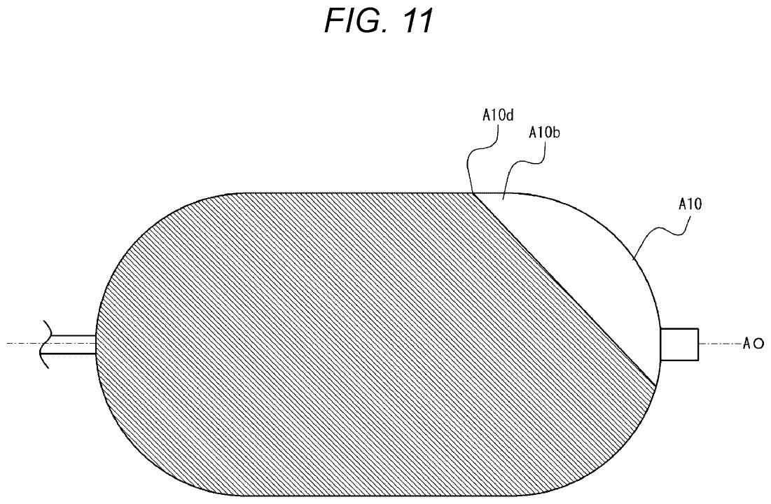

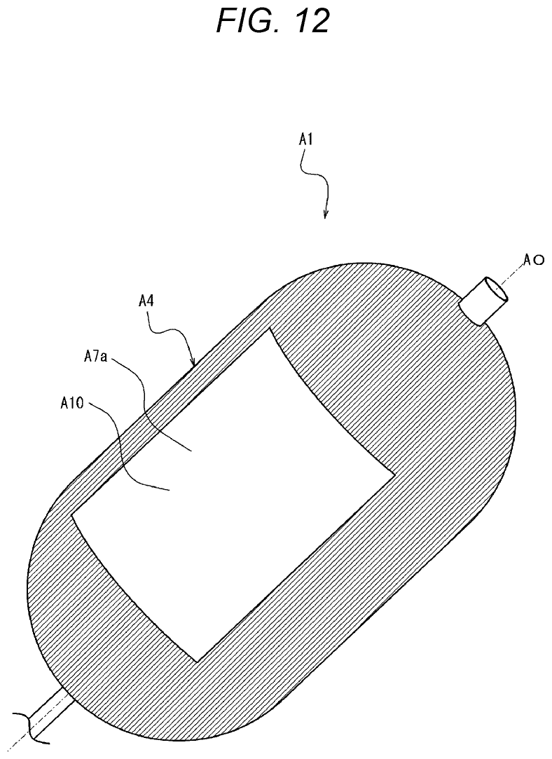

As in the third embodiment illustrated in FIGS. 10 and 11, the window A10 may be configured such that the tip end A10d of the proximal end portion A10b of the window A10 is located on the distal end side with respect to the center of the enclosure A4 in the longitudinal direction. According to such a configuration, for example, by using another phototherapy device A1 for radial irradiation having the window A10 only on the peripheral wall A7a as illustrated in FIG. 12 in combination, it is possible to enable efficient treatment in a certain case of gynecological cancer. The angular range in which the window A10 in FIG. 12 is disposed can be, for example, preferably a range of 45 degrees to 270 degrees, more specifically, a range of 90 degrees to 180 degrees in the circumferential direction of the axial line AO.

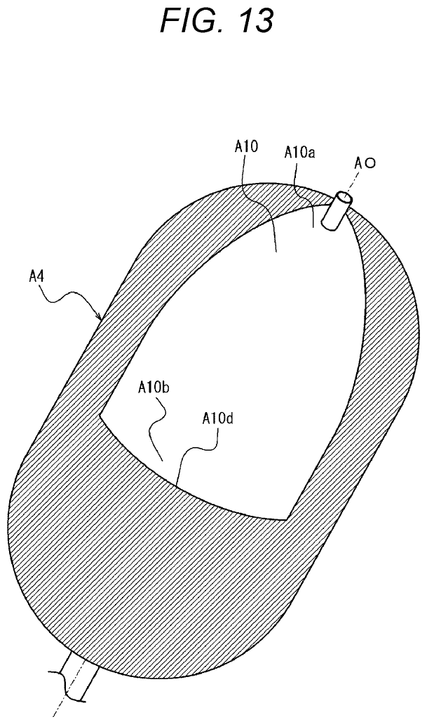

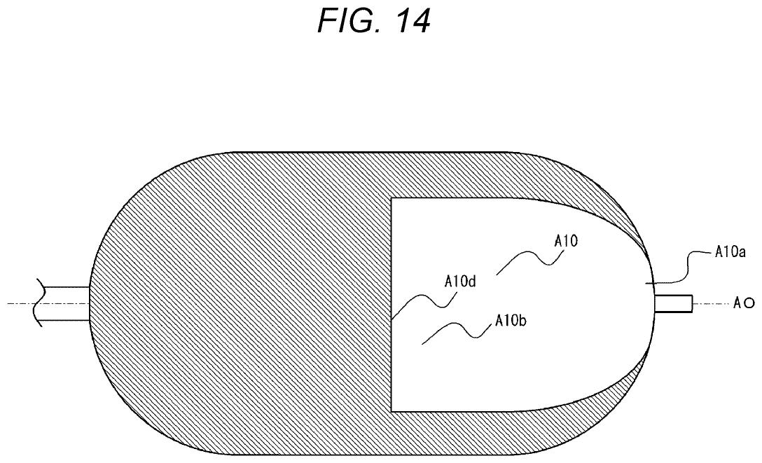

As in the fourth embodiment illustrated in FIGS. 13 and 14, the window A10 may be configured such that the angular range in which the window A10 is disposed is constant from the distal end portion A10a of the window A10 to the tip end A10d of the proximal end portion A10b. The angular range in which in the circumferential direction of the axial line AO on which the window A10 is disposed can be, for example, a range of 45 degrees to 180 degrees, more specifically a range of 60 degrees to 120 degrees, and preferably 90 degrees. According to such a configuration, light can be emitted with a constant width in the circumferential direction from the distal end portion A10a of the window A10 to the tip end A10d of the proximal end portion A10b, so that it is possible to efficiently treat a certain case of gynecological cancer.

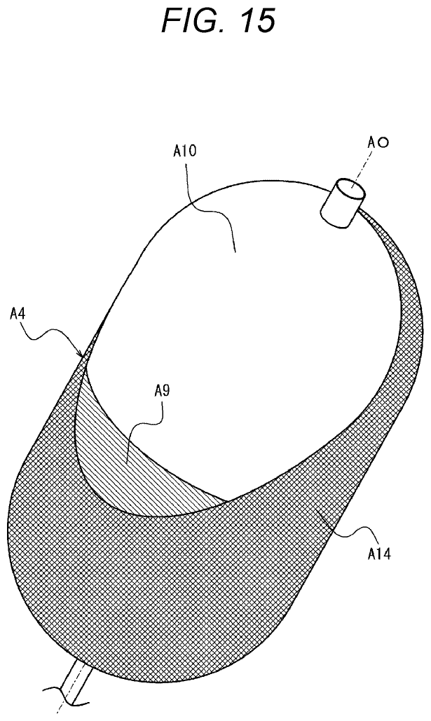

As in the fifth embodiment illustrated in FIG. 15, the enclosure A4 may include an absorber A14 that at least partially absorbs light that is not blocked by the light-blocking body A9 but partially passes through the light-blocking body A9, or light that is emitted from the light emitter A2a, passes through the enclosure A4, and then enters the enclosure A4 from outside (for example, light transmitted through the window A10). The absorber A14 is made of a light absorbing material. Examples of the light absorbing material include graphite; carbon black; oxide-based black pigment such as triiron tetraoxide, copper-chromium-zinc composite oxide, and the like; or various pigments that absorb a specific wavelength region can be used. The absorber A14 may be configured to cover a part of the outer surface of the light-blocking body A9 as illustrated, or may be configured to cover the entire outer surface of the light-blocking body A9. According to the absorber A14, it is possible to easily suppress incidence of light on a normal tissue due to light blocking outside the enclosure A4. The thickness of the absorber A14 can be, for example, preferably in a range of 1 μm to 100 μm, more specifically in a range of 5 μm to 35 μm.

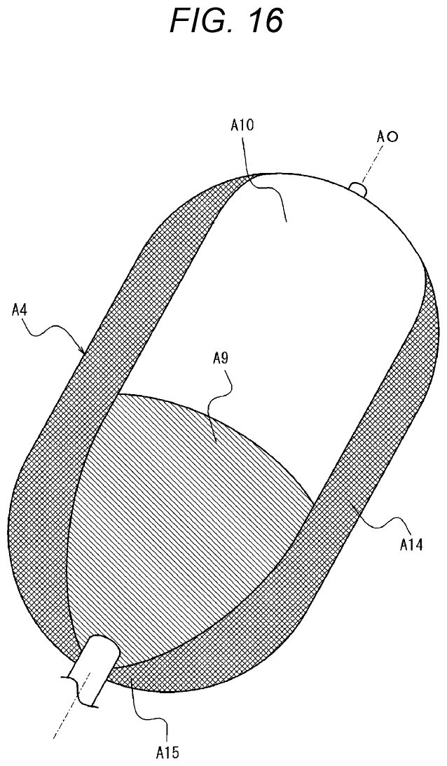

As in the sixth embodiment illustrated in FIG. 16, the enclosure A4 may have a configuration in which the proximal end portion A4b of the enclosure A4 has a labeling body A15 indicating the orientation of the window A10 in the circumferential direction. The labeling body A15 may be formed by, for example, a colored portion that covers a part of the outer surface of the proximal end portion A4b of the enclosure A4 with a predetermined color. The labeling body A15 may be formed at the proximal end portion of the absorber A14. In the illustrated example, the absorber A14 is disposed across from the distal end portion A4a to the proximal end portion A4b of the enclosure A4, and the angular range in which the absorber A14 is disposed is set so as to avoid the window A10 and to be constant from the distal end portion A4a to the proximal end portion A4b of the enclosure A4. According to the labeling body A15, when the abnormal tissue is irradiated with light through the window A10, it is possible to easily confirm the circumferential position of the window A10 by visually recognizing the labeling body A15.

The phototherapy method according to an embodiment as described below may be performed using the phototherapy device A1 according to any one or some of the embodiments described above.

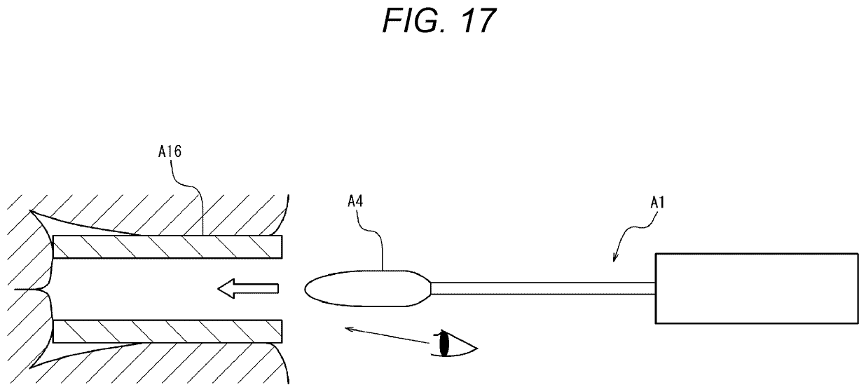

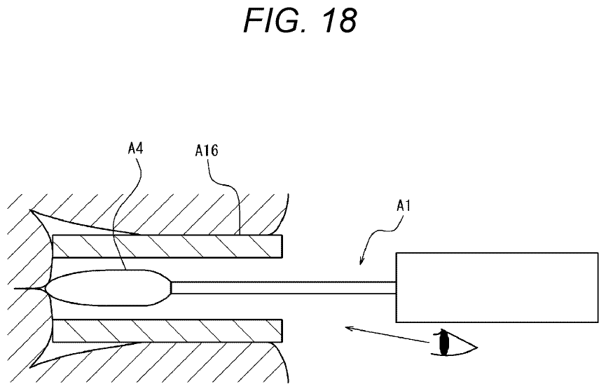

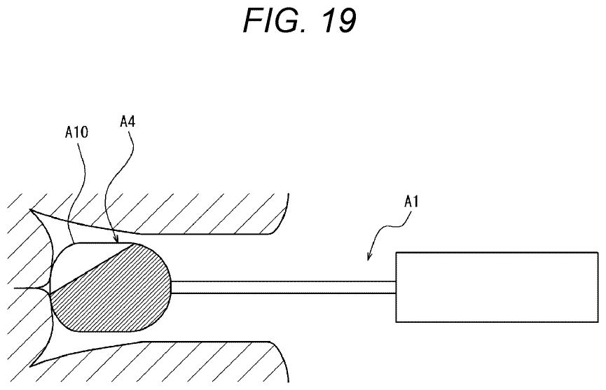

The phototherapy method according to the present embodiment includes an insertion step AS1 of inserting the enclosure A4 into the living body as illustrated in FIGS. 17 and 18, a rotation step AS2 of rotating the enclosure A4 in the circumferential direction in accordance with the position of the tumor, an emission step AS3 of emitting light from the enclosure A4 through the window A10 as illustrated in FIG. 19, and a removal step AS4 of removing the enclosure A4 from the living body.

Note that the removal step AS4 is performed after the insertion step AS1, and the rotation step AS2 and the emission step AS3 are performed before the removal step AS4 is completed. The rotation step AS2 may be performed before the insertion step AS1 or may be performed after the insertion step AS1. For example, the insertion step AS1, the rotation step AS2, the emission step AS3, and the removal step AS4 may be performed in this order, or the rotation step AS2, the insertion step AS1, the emission step AS3, and the removal step AS4 may be performed in this order. In addition, an administration step of administering the antibody-photosensitizer complex (substance containing photosensitizer) to the patient may be included before the insertion step AS1. Here, the photosensitizer can be, for example, a substance configured to contain a phthalocyanine dye. The phthalocyanine dye can be, for example, IR700 (infrared phthalocyanine dye). Alternatively, it may have an administration step of administering the antibody-photosensitizer to the patient prior to the insertion step AS1. The antibody that binds the phthalocyanine dye is a monoclonal antibody that targets cell surface receptors, such as cetuximab, a chimeric anti-human epidermal growth factor receptor (EGFR) monoclonal antibody (IgG1). The antibody-photosensitizer complex is an antibody-photosensitizer complex in which cetuximab and a dye IR700 as a photosensitizer are bound, or an antibody-photosensitizer complex in which a monoclonal antibody (anti-CD25 antibody) that specifically binds to an interleukin 2 (IL-2) receptor a chain (CD25) on the cell surface and a dye IR700 as a photosensitizer are bound.

In the present embodiment, the living body is a vaginal cavity. The phototherapy method according to the present embodiment may be applied to a living body other than the vaginal cavity.

The phototherapy method according to the present embodiment includes an expansion step AS5 of expanding the balloon as the enclosure A4. Note that, in the phototherapy method according to the present embodiment, the enclosure A4 may not be configured as a balloon, but may be configured to always have a mode corresponding to the expanded mode, and may be configured not to have the expansion step AS5.

The phototherapy method according to the present embodiment may include a contraction step AS6 of contracting the balloon, and the contraction step AS6 may be performed after the emission step AS3 and before the removal step AS4.

The phototherapy method according to the present embodiment includes a visual recognition step AS7 of visually recognizing the uterine orifice and the affected area with a colposcope or the other visual recognition instrument A16, and a visual recognition instrument removal step AS8 of removing the visual recognition instrument A16 from the inside of the vagina. The visual recognition step AS7 is performed before the insertion step AS1, the rotation step AS2, the emission step AS3, the removal step AS4, and the expansion step AS5, and the visual recognition instrument removal step AS8 is performed after the insertion step AS1 and before the emission step AS3. Note that the phototherapy method according to the present embodiment may be configured not to include the visual recognition step AS7 and the visual recognition instrument removal step AS8.

The phototherapy method according to the present embodiment includes a confirmation step AS9 of confirming that the enclosure A4 has been abutted against the deepest part of the vagina by the insertion step AS1. Note that the phototherapy method according to the present embodiment may be configured not to include the confirmation step AS9.

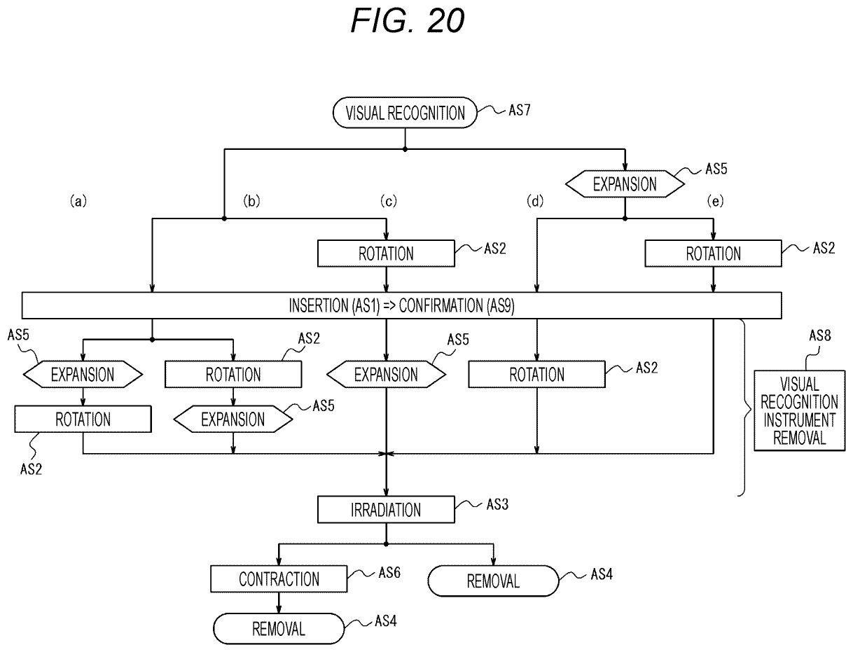

The phototherapy method according to the present embodiment may be configured to perform the insertion step AS1, the expansion step AS5, the rotation step AS2, the emission step AS3, and the removal step AS4 in this order as illustrated as a pattern (a) in FIG. 20.

The phototherapy method according to the present embodiment may be configured to perform the insertion step AS1, the rotation step AS2, the expansion step AS5, the emission step AS3, and the removal step AS4 in this order as illustrated as a pattern (b) in FIG. 20.

The phototherapy method according to the present embodiment may be configured to perform the rotation step AS2, the insertion step AS1, the expansion step AS5, the emission step AS3, and the removal step AS4 in this order as illustrated as a pattern (c) in FIG. 20.

The phototherapy method according to the present embodiment may be configured to perform the expansion step AS5, the insertion step AS1, the rotation step AS2, the emission step AS3, and the removal step AS4 in this order as illustrated as a pattern (d) in FIG. 20.

The phototherapy method according to the present embodiment may be configured to perform the expansion step AS5, the rotation step AS2, the insertion step AS1, the emission step AS3, and the removal step AS4 in this order as illustrated as a pattern (e) in FIG. 20.

The phototherapy method according to the present embodiment may include a sheath removal step of removing the protective sheath from inside the living body, perform the insertion step AS1 in a state where the balloon in the contracted mode is protected by the protective sheath, perform the sheath removal step after the insertion step AS1, and perform the expansion step AS5 after the sheath removal step.

The phototherapy method according to the present embodiment may include an entire (or whole) irradiation step of irradiating the entire (or whole) inside of the living body with light having output power smaller than the output power in the emission step AS3, and the entire (or whole) irradiation step may be performed after the emission step AS3.

The phototherapy method according to the present embodiment may include an expansion confirmation step of confirming using ultrasound that the balloon has been expanded, perform the expansion step AS5 after the insertion step AS1, and perform the expansion confirmation step after the expansion step AS5.

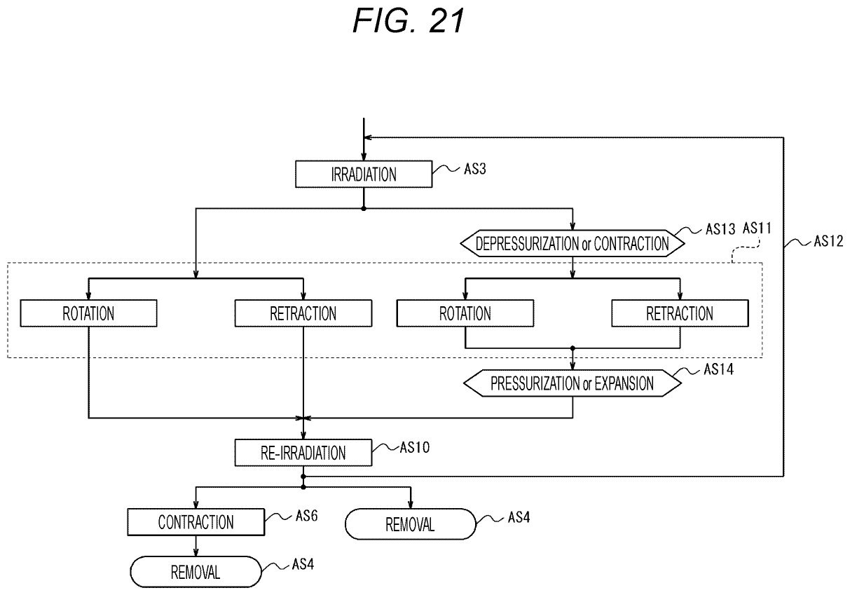

As illustrated in FIG. 21, the phototherapy method according to the present embodiment may include a re-emission step AS10 of re-emitting light by changing the position of the emission range in the living body after the emission step AS3.

The phototherapy method according to the present embodiment may include a position changing step AS11 of rotating the enclosure A4 in the circumferential direction or moving the enclosure A4 in the longitudinal direction (for example, retracting from the far side to the near side), and the position changing step AS11 may be performed after the emission step AS3 and before the re-emission step AS10. The phototherapy method according to the present embodiment may include a depressurization/contraction step AS13 of depressurizing or contracting the balloon and a pressurization/expansion step AS14 of pressurizing or expanding the balloon. The depressurization/contraction step AS13 may be performed after the emission step AS3 and before the position changing step AS11, and the pressurization/expansion step AS14 may be performed after the position changing step AS11 and before the re-emission step AS10.

The phototherapy method according to the present embodiment may include a repetitive emission step AS12 in which the position changing step AS11 and the re-emission step AS10 are repeated a plurality of times in this order after the emission step AS3. The repetitive emission step AS12 may be performed as necessary.

The phototherapy method according to the present embodiment uses (i) a first phototherapy device A1 (for example, the phototherapy device A1 illustrated in FIG. 11) as a phototherapy device A1, the first phototherapy device A1 including a first enclosure A4 enclosing a first internal region A3 in which a first light emitter A2a is disposed and defining a first longitudinal direction and a first axial line AO extending in the first longitudinal direction, the first enclosure A4 including a first light-blocking body A9 that blocks at least a part of the first light emitted from the first light emitter A2a from being transmitted toward the outside of the first internal region A3 and a first window A10 that transmits the first light toward the outside of the first internal region A3 due to absence of the first light-blocking body A9, the first window A10 extending from one end portion A10a of the first window A10 disposed in one end portion A4a of the first enclosure A4 in the first longitudinal direction toward the other end portion A4b of the first enclosure A4, and the other end portion A10b of the first window A10 in the first longitudinal direction being disposed over a limited angular range not extending over the entire periphery in the circumferential direction of the first axial line AO, and (ii) a second phototherapy device A1 (for example, the phototherapy device A1 illustrated in FIG. 12) including a second enclosure A4 enclosing a second internal region A3 in which a second light emitter A2a and defining a second longitudinal direction and a second axial line AO extending in the second longitudinal direction, the second enclosure A4 including a second light-blocking body A9 blocking at least a part of second light emitted from the second light emitter A2a from being transmitted toward the outside of the second internal region A3 and a second window A10 transmitting the second light toward the outside of the second internal region A3 due to absence of the second light-blocking body A9, and the second window A10 being disposed in a portion other than the both end portions A4a and A4b of the second enclosure A4 in the second longitudinal direction. The phototherapy method may include (iii) a first phototherapy device insertion step AS1 of inserting the first enclosure A4 into the living body as the insertion step AS1, a first phototherapy device rotation step AS2 of rotating the first enclosure A4 in a first circumferential direction in accordance with the position of a tumor as the rotation step AS2, a first phototherapy device emission step AS3 of irradiating the first light from the first enclosure A4 through the first window A10 as the emission step AS3, a first phototherapy device removal step AS4 of removing the first enclosure A4 from the living body as the removal step AS4, (iv) a second phototherapy device insertion step of inserting the second enclosure A4 into the living body, a second phototherapy device rotation step of rotating the second enclosure A4 in the second circumferential direction in accordance with the position of the tumor, a second phototherapy device emission step of irradiating the second light from the second enclosure A4 through the second window A10, and a second phototherapy device removal step of removing the second enclosure A4 from the living body. The first light-blocking body A9 may be configured as a first reflector that at least partially reflects the first light emitted from the first light emitter A2a toward the first internal region A3. The second light-blocking body A9 may be configured as a second reflector that at least partially reflects the second light emitted from the second light emitter A2a toward the second internal region A3.

The phototherapy method according to the present embodiment may be configured to perform the second phototherapy device insertion step, the second phototherapy device rotation step, the second phototherapy device emission step, and the second phototherapy device removal step after the first phototherapy device insertion step AS1, the first phototherapy device rotation step AS2, the first phototherapy device emission step AS3, and the first phototherapy device removal step AS4. The phototherapy method according to the present embodiment may include a visual recognition instrument insertion step of inserting the colposcope or the other visual recognition instrument A16 into the living body, and the visual recognition instrument insertion step may be performed after the first phototherapy device emission step AS3 and before the first phototherapy device removal step AS4, or after the first phototherapy device removal step AS4 and before the second phototherapy device insertion step.

The phototherapy method according to the present embodiment may be configured to perform the first phototherapy device insertion step AS1, the first phototherapy device rotation step AS2, the first phototherapy device emission step AS3, and the first phototherapy device removal step AS4 after the second phototherapy device insertion step, the second phototherapy device rotation step, the second phototherapy device emission step, and the second phototherapy device removal step. The phototherapy method according to the present embodiment may include a visual recognition instrument insertion step of inserting the colposcope or the other visual recognition instrument A16 into the living body, and the visual recognition instrument insertion step may be performed after the second phototherapy device emission step and before the second phototherapy device removal step, or after the second phototherapy device removal step and before the first phototherapy device insertion step AS1. In the case of irradiating the affected area long in the longitudinal direction (longer than the length of the window A10 in the longitudinal direction), the irradiation in the second phototherapy device emission step may be performed on a site close to the hand, and the first phototherapy device emission step AS3 may be performed after it is confirmed in the confirmation step AS9 that the first enclosure A4 has been abutted against the deepest part of the vagina.

The phototherapy method according to the present embodiment may include a second phototherapy device expansion step of expanding the second balloon as the second enclosure. The phototherapy method according to the present embodiment may include a second phototherapy device contracting step of contracting the second balloon, and may be configured to perform the second phototherapy device contracting step after the second phototherapy device emission step and before the second phototherapy device removing step.

The phototherapy method according to the present embodiment may include a liquid replacement step of replacing the air in the balloon (enclosure A4) with a liquid such as physiological saline as fluid for expanding the balloon, and the liquid replacement step may be performed before the expansion step AS5.

The phototherapy method according to the present embodiment may include a fixing step of fixing the enclosure A4 at a determined position until the emission step AS3 ends after the position of the enclosure A4 is determined in the rotation step AS2.

The phototherapy method according to the present embodiment may include a colposcope rotation step of rotating the colposcope so that the affected area can be seen from the gap of the colposcope. In the rotation step AS2, the enclosure A4 may be rotated according to the angle of the colposcope. In this case, the hand of the colposcope may be marked to indicate the angle of the affected area. The structure itself of the colposcope, such as a hinge or a grip of the colposcope, may be used as a mark for rotating the enclosure A4.

In the confirmation step AS9, it may be confirmed that the enclosure A4 has been abutted against the deepest part of the vagina using ultrasound or an endoscope.

Using ultrasound, an endoscope, computed tomography (CT), or magnetic resonance imaging (MRI), it may be confirmed that the tumor is located within the irradiation range.

The first aspect of the present disclosure is not limited to the above-described embodiment and may be modified in various manners without departing from the gist of the present disclosure.

Therefore, the phototherapy device A1 according to the embodiment of the first aspect described above can be variously changed and includes the enclosure A4 that forms the internal region A3 in which the light emitter A2a is disposed and defines the longitudinal direction and the axial line AO extending in the longitudinal direction, the enclosure A4 includes the light-blocking body A9 that blocks at least a part of the light emitted from the light emitter A2a from being transmitted toward the outside of the internal region A3 and the window A10 that transmits the light toward the outside of the internal region A3 due to the absence of the light-blocking body A9, the window A10 extends from one end portion of the window A10 located at one end portion of the enclosure A4 in the longitudinal direction toward the other end portion of the enclosure A4, and the other end portion of the window A10 in the longitudinal direction includes is disposed over a limited angular range not extending over the entire periphery in the circumferential direction of the axial line AO.

For example, the phototherapy device A1 is not limited to the configuration in which the light emitter A2a is disposed on the axial line AO in the internal region A3 of the enclosure A4. The light emitter A2a is not limited to the configuration capable of emitting light toward the entire region in the circumferential direction of the axial center, and may be configured to emit light toward only a partial region in the circumferential direction of the axial center, for example. The phototherapy device A1 is not limited to the configuration in which the light emitter A2a is configured to be introduced into the internal region A3 of the enclosure A4 by moving in the optical lumen A5, and for example, the light emitter A2a may be incorporated in the internal region A3 of the enclosure A4.

Note that the phototherapy device A1 according to the above-described embodiment is preferably the phototherapy device A1 in which the tip end A10c of the one end portion of the window A10 is located at the tip end A4c of the one end portion of the enclosure A4.

The phototherapy device A1 according to the above-described embodiment is preferably a phototherapy device A1 having a lumen A5 serving as a movement path of the light emitter A2a.

The phototherapy device A1 according to the above-described embodiment is preferably a phototherapy device A1 in which the light emitter A2a is introduced into the internal region A3 through the other end portion of the enclosure A4 in the lumen A5 serving as a movement path of the light emitter A2a.

The phototherapy device A1 according to the above-described embodiment is preferably a phototherapy device A1 in which the other end portion of the window A10 is defined by a straight line perpendicular to the longitudinal direction.

The phototherapy device A1 according to the above-described embodiment is preferably a phototherapy device A1 in which the enclosure A4 includes the absorber A14 that at least partially absorbs light that is emitted from the light emitter A2a, passes through the enclosure A4, and then enters the enclosure A4 from outside.

The phototherapy device A1 according to the above-described embodiment is preferably a phototherapy device A1 in which the other end portion of the enclosure A4 includes a labeling body A15 indicating the orientation of the window A10 in the circumferential direction.

The phototherapy device A1 according to the above-described embodiment is preferably a phototherapy device A1 in which the enclosure A4 is a balloon that is configured to be deformable from the contracted mode to the expanded mode.

The phototherapy device A1 according to the above-described embodiment is preferably a phototherapy device A1 that includes a lumen A6 serving as a movement path of fluid that deforms the enclosure A4 from the contracted mode to the expanded mode.

The phototherapy device A1 according to the above-described embodiment is preferably a phototherapy device A1 in which fluid is introduced into the internal region A3 through the other end portion of the enclosure A4 in the lumen A6 serving as a fluid movement path.

The phototherapy device A1 according to the above-described embodiment is preferably used in the phototherapy method according to the above-described embodiment.

Next, an embodiment of a second aspect of the present disclosure will be described. A phototherapy device B1 according to an embodiment of the second aspect of the present disclosure includes: an enclosure (balloon B4) that forms an internal region (internal space B3) in which a light emitter B2a is disposed and defines a longitudinal direction and an axial line BO extending in the longitudinal direction; and a lumen B6 serving as a movement path of fluid that deforms the enclosure from a contracted mode to an expanded mode. The enclosure can include: a light-blocking body (reflector 9) that blocks at least part of light emitted from the light emitter B2a from being transmitted toward the outside of the internal region; and a window B10 that transmits light toward the outside of the internal region due to absence of the light-blocking body. The window B10 may extend from one end portion of the window B10 located at one end portion of the enclosure in the longitudinal direction toward the other end portion of the enclosure. The other end portion of the window B10 in the longitudinal direction may be disposed over a limited angular range not extending over the entire periphery in the circumferential direction of the axial line BO. The light emitter B2a may be disposed on the axial line in the inner region. The light-blocking body may be provided at least at one end portion of the enclosure in the longitudinal direction so as to have different thicknesses depending on a position, but the present invention is not limited thereto.

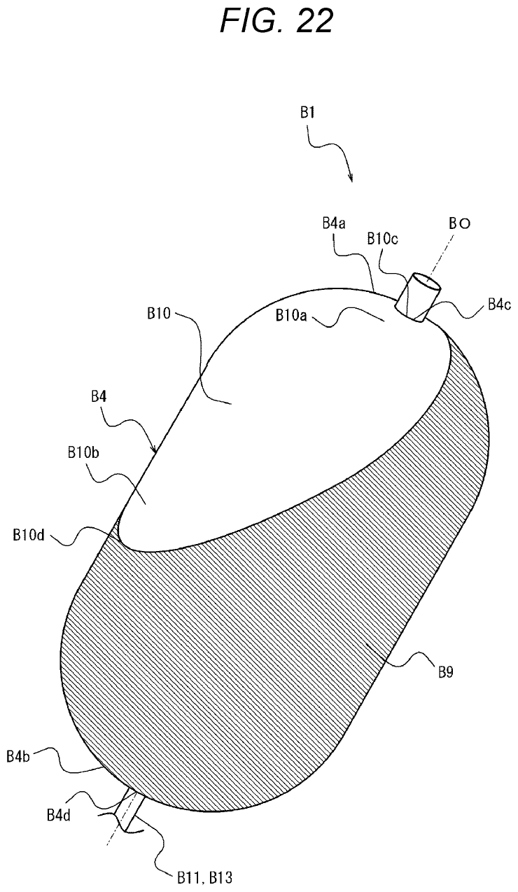

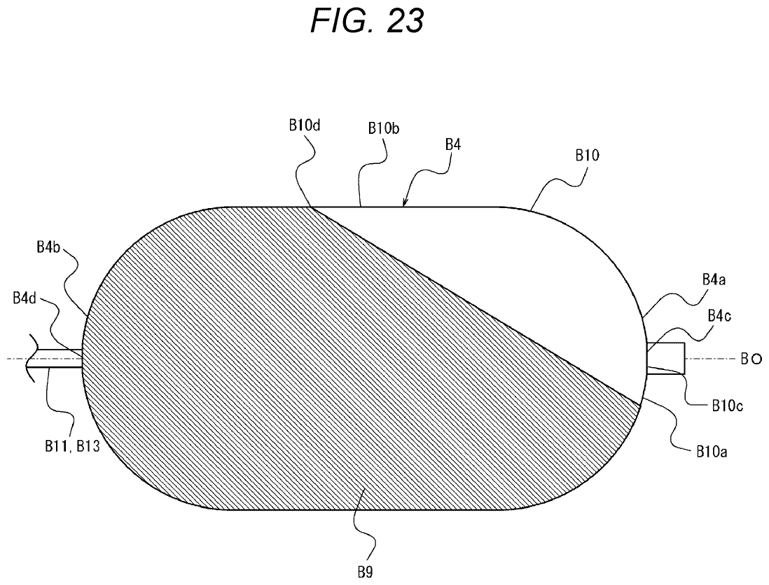

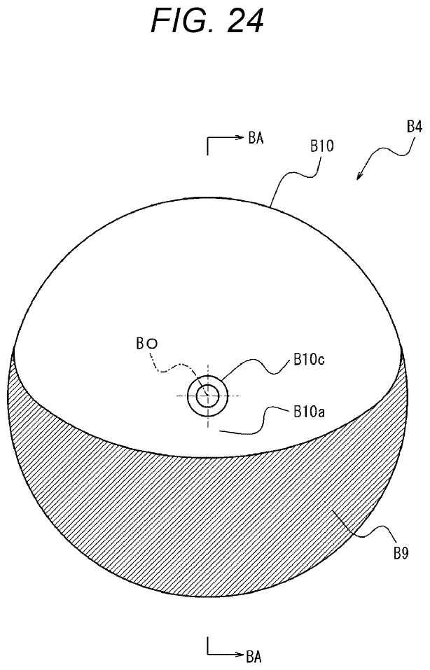

A phototherapy device B1 according to an embodiment of the second aspect of the present disclosure illustrated in FIGS. 22 to 27 is used to irradiate an abnormal tissue in a living body with therapeutic light in a phototherapeutic treatment such as photoimmunotherapy (PIT) or photodynamic therapy (PDT). The light can be, for example, laser light.

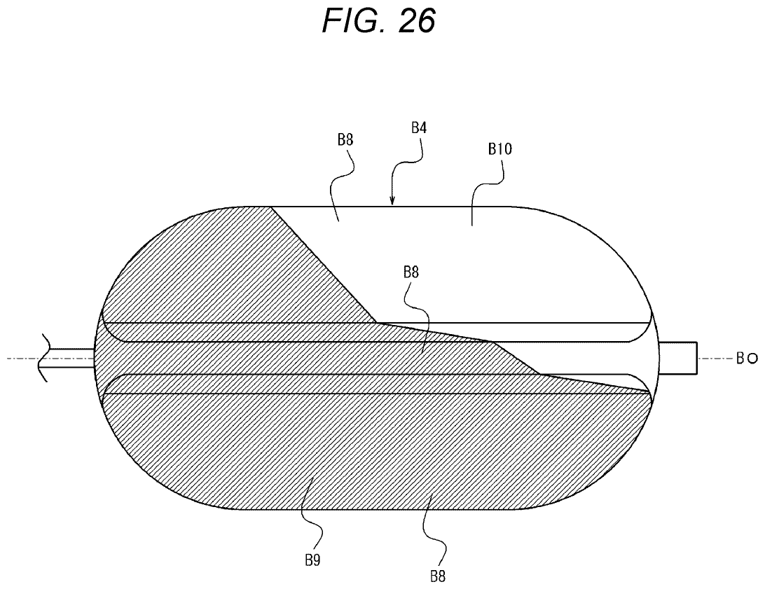

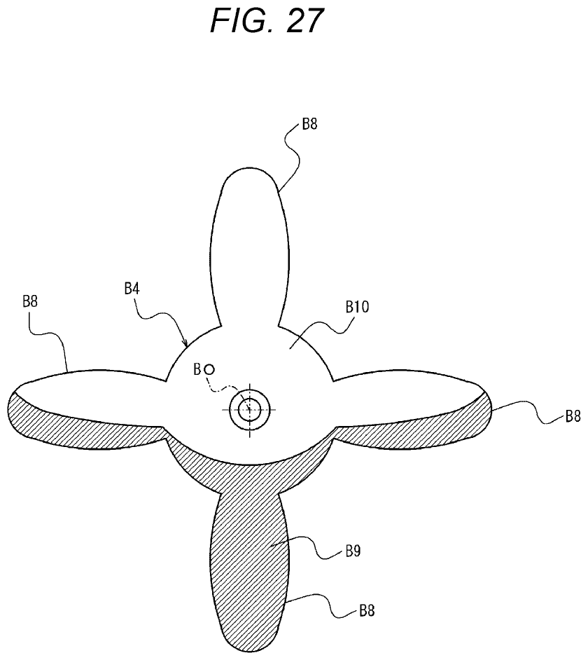

The phototherapy device B1 includes a light emitting device B2 having a light emitter B2a, a balloon B4 forming an internal space B3 in which the light emitter B2a is disposed and defining a longitudinal direction and an axial line BO extending in the longitudinal direction, a lumen B5 (hereinafter, also referred to as an optical lumen B5) serving as a movement path of the light emitter B2a, and a lumen B6 (hereinafter, the fluid lumen is also referred to as a fluid lumen B6) serving as a movement path of fluid that deforms the balloon B4 from the contracted mode illustrated in FIGS. 26 and 27 to the expanded mode illustrated in FIGS. 22 to 25. The balloon B4 can be deformed from the contracted mode to the expanded mode by the pressure of the fluid moving within the fluid lumen B6 and entering the internal space B3.

Note that a direction along a straight line perpendicular to the axial line BO is also referred to as a radial direction or simply a radial direction of the axial line BO, and a direction around the axial line BO is also referred to as a circumferential direction or simply a circumferential direction of the axial line BO. In the present embodiment, the axial line BO is the central axial line of the balloon B4. The axial line BO is not limited to the central axis.

The balloon B4 is elongated in a predetermined direction in the expanded mode, and defines a longitudinal direction as the predetermined direction. In addition, the balloon B4 has a tubular shape in which both end portions in the longitudinal direction are occluded around the axial line BO. The light emitter B2a is disposed at a predetermined position on the axial line BO in the internal space B3. The arrangement of the light emitter B2a in the longitudinal direction in the internal space B3 can be appropriately set.

In the expanded mode, the balloon B4 has a cylindrical shape with both end portions in the longitudinal direction occluded around the axial line BO. More specifically, the balloon B4 includes a peripheral wall B7a having a cylindrical shape in the expanded mode, one end wall B7b having a convex curved shape continuous with one end portion of the peripheral wall B7a and protruding outward in the longitudinal direction in the expanded mode, and another end wall B7b having a convex curved shape continuous with the other end portion of the peripheral wall B7a and protruding outward in the longitudinal direction in the expanded mode.

Both or any one of the end walls B7b of the balloon B4 may have a convex shape other than a convex curved shape in the expanded mode. The balloon B4 may have a configuration in which the peripheral wall B7a has a tubular shape other than a cylindrical shape, such as an elliptical cylindrical shape in the expanded mode. That is, the balloon B4 may have a tubular shape other than a cylindrical shape in which both end portions in the longitudinal direction are closed around the axial line BO in the expanded mode.

The contracted mode is a mode in which the balloon B4 is folded into a predetermined shape so that the internal space B3 is reduced from the expanded mode. The predetermined shape may be a shape in which a plurality of pleats B8 are arranged in the circumferential direction, and may be, for example, a shape in which four pleats B8 are formed as illustrated in FIGS. 26 and 27.

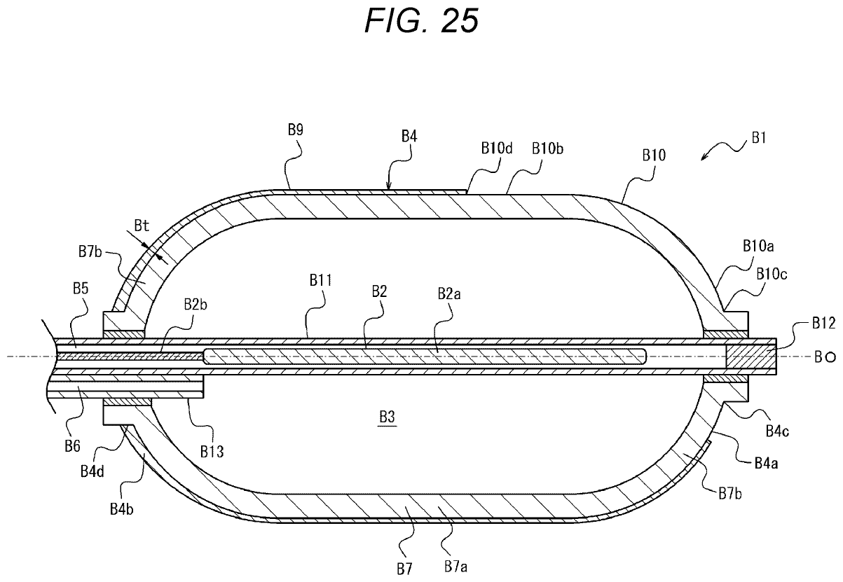

As illustrated in FIG. 25, the light emitting device B2 further includes a light source and an elongated light guide unit B2b that transmits the light emitted from the light source to the light emitter B2a. The light guide unit B2b is configured by, for example, a light guide such as an optical fiber, and is continuous with the light source at one end portion and is continuous with the light emitter B2a at the other end portion. In addition, the light guide unit B2b may have an optical connector that is connected to the light guide and is configured to be detachable from the light source.

The light emitter B2a extends in an elongated shape along the axial center, and is configured to be capable of emitting light transmitted from the light source via the light guide unit B2b in the radial direction of the axial center from a portion over a predetermined width in the direction along the axial center. More specifically, the light emitter B2a can emit light toward the entire region in the circumferential direction of the axial center.

The balloon B4 includes a light-blocking body B9 that blocks at least part of light emitted from the light emitter B2a from being transmitted toward the outside of the internal space B3, and a window B10 that transmits light toward the outside of the internal space B3 due to absence of the light-blocking body B9. More specifically, the balloon B4 includes a main body B7 having a peripheral wall B7a and two end walls B7b, and a light-blocking body B9 covering a part of an outer surface of the main body B7, and the window B10 is configured by a portion of the main body B7 that is not covered with the light-blocking body B9. The main body B7 is formed of a light transmitting material, and the light-blocking body B9 is formed of a light blocking material. The light-blocking body B9 may be configured as a reflector that at least partially reflects the light emitted from the light emitter B2a toward the internal region (internal space B3). In this case, the light-blocking body B9 is formed of a light reflective material. The main body B7 can be made of, for example, a resin material, and is preferably made of polyethylene terephthalate, polyurethane, nylon, or the like. The light-blocking body B9 can be formed of, for example, various metal materials (for example, titanium oxide, barium sulfate, zinc oxide, and the like). According to the light-blocking body B9, the light emitted from the light emitter B2a can be collected and efficiently emitted to a limited range through the window B10.

The light-blocking body B9 is disposed at least at one end portion (hereinafter, also referred to as a proximal end portion B4b) of the balloon B4 in the longitudinal direction such that a thickness Bt varies depending on the position. In the present embodiment, as illustrated in FIG. 25, the light-blocking body B9 is disposed such that the thickness Bt gradually decreases as the position changes radially outward of the axial line BO. The thickness Bt is preferably in a range of 1 μm to 500 μm, more specifically in a range of 10 μm to 450 μm. As illustrated in FIG. 25 and the like, the light-blocking body B9 may also be disposed at the other end portion (hereinafter, also referred to as a distal end portion B4a) of the balloon B4 in the longitudinal direction. Furthermore, as illustrated in FIG. 25 and the like, the light-blocking body B9 may also be disposed in a portion excluding both end portions of the balloon B4 in the longitudinal direction so as to form the window B10.

The method for forming the light-blocking body B9 is not particularly limited, and the light-blocking body B9 can be formed by, for example, coating to the main body B7. The coating method is not particularly limited, and for example, a dipping method, a spray coating method, a roll coating method, a screen printing method, or the like can be used. In particular, according to the dipping method, the light-blocking body B9 can be easily formed such that the thickness Bt of the light-blocking body B9 decreases toward the radial outside of the axial line BO. The light-blocking body B9 is not limited to cover a part of the outer surface of the main body B7, but may cover a part of the inner surface of the main body B7, for example.

The arrangement and shape of the window B10 are not particularly limited, and the window B10 may be configured to extend from, for example, one end portion (hereinafter, also referred to as a distal end portion B10a) of the window B10 located at the distal end portion B4a of the balloon B4 in the longitudinal direction toward the proximal end portion B4b of the balloon B4 as illustrated in the drawing. In addition, as illustrated in the drawing, a tip end B10c of the distal end portion B10a of the window B10 may be located at a tip end B4c of the distal end portion B4a of the balloon B4. That is, the window B10 may be disposed in a region including the center of the balloon B4 as viewed in the longitudinal direction.

The other end portion (hereinafter, also referred to as a proximal end portion B10b) of the window B10 in the longitudinal direction may be disposed over a limited angular range not extending over the entire periphery in the circumferential direction of the axial line BO. That is, the proximal end portion B10b of the window B10 may be disposed only in a part in the circumferential direction. An angular range in which the window B10 is disposed may be set so as to be gradually narrowed from the distal end portion B10a toward the proximal end portion B10b of the window B10.

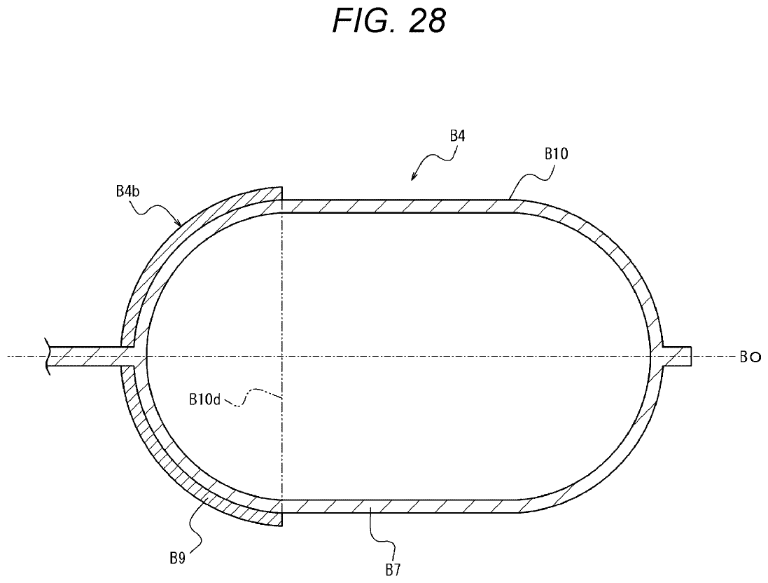

A tip end B10d of the proximal end portion B10b of the window B10 in the longitudinal direction may be disposed in a range over the entire periphery in the circumferential direction of the axial line BO. For example, as illustrated in FIG. 28, the window B10 may be disposed over the entire periphery of the entire portion excluding the proximal end portion B4b of the balloon B4.

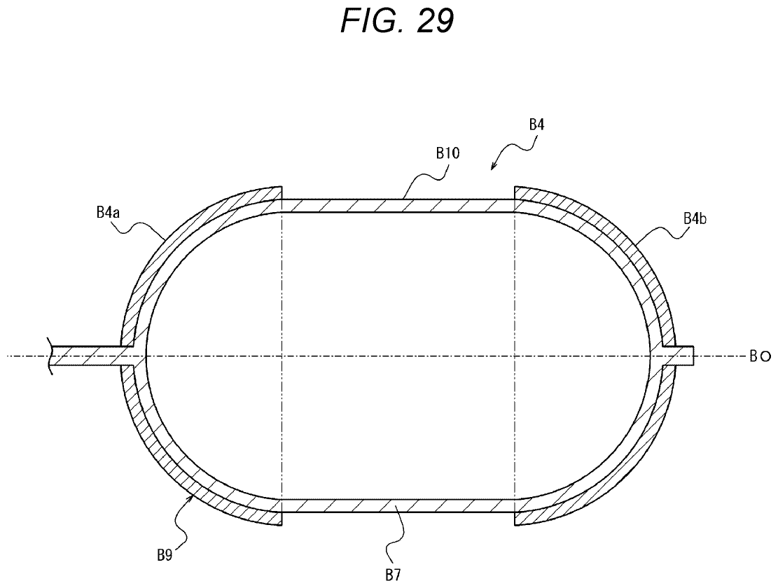

As illustrated in FIG. 29, the window B10 may be disposed only in a portion between the distal end portion B4a and the proximal end portion B4b of the balloon B4 over the entire periphery (or only in a part in the circumferential direction).

The light emitter B2a is introduced into the internal space B3 through a tip end B4d of the proximal end portion B4b of the balloon B4 within the optical lumen B5. The optical lumen B5 is formed by a tube B11 (hereinafter, also referred to as an optical tube B11) arranged coaxially with the axial line BO, wherein the optical tube B11 passes through a proximal end portion B4b and a distal end portion B4a of the balloon B4. More specifically, the optical tube B11 is bonded to the end wall B7b at the proximal end portion B4b of the balloon B4, and bonded to the end wall B7b at the distal end portion B4a of the balloon B4. The optical tube B11 is closed by a closing member B12 at the distal end portion B4a of the balloon B4. The optical tube B11 has optical transparency.

The optical tube B11 may have a marker such as a contrast marker having radiopacity serving as a mark for arranging the light emitter B2a at the predetermined position. Alternatively, the optical tube B11 may have a stopper that restricts the movement of the light emitter B2a in the introduction direction in the optical lumen B5 by hitting the light emitter B2a at the predetermined position. The stopper may be a diameter-reduced portion disposed in the optical tube B11, or may be a member disposed in the optical tube B11.

Fluid is introduced into the internal space B3 through the proximal end portion B4b of the balloon B4 within the fluid lumen B6 to deform the balloon B4 from a contracted mode to the expanded mode. The fluid lumen B6 is formed by a tube B13 (hereinafter, also referred to as a fluid tube B13) disposed adjacent to and in parallel with the optical tube B11, and the fluid tube B13 passes through the proximal end portion B4b of the balloon B4. More specifically, the fluid tube B13 is adhered to the end wall B7b at the proximal end portion B4b of the balloon B4. The fluid tube B13 opens in the internal space B3.

The fluid lumen B6 is not limited to the configuration disposed in parallel with the optical lumen B5, and for example, may be disposed on the radial outside of the optical lumen B5 coaxially with the optical lumen B5, or may be configured with a common lumen that is configured to be used as both the fluid lumen B6 and the optical lumen B5.

According to the phototherapy device B1 of the present embodiment, for example, in the case of treating gynecological cancer, the balloon B4 is deformed from the contracted mode to the expanded mode in front of the uterine orifice, and light can be widely emitted from the distal end portion B10a of the window B10 to the vicinity of the uterine orifice, and at the same time, light can be limitedly emitted to the vaginal wall from the remaining portion of the window B10 in the circumferential direction. Therefore, in certain cases of gynecological cancer, it is possible to safely treat an abnormal site while suppressing irradiation of a normal site of the vaginal wall with light. Note that the phototherapy device B1 may be applied to cancer other than gynecological cancer (for example, esophageal cancer or gastric cancer). For example, in a case where the phototherapy device B1 is applied to a hollow organ such as a digestive tract (esophagus, stomach, small intestine, large intestine, etc.), a urinary tract, and a blood vessel as a living body, the phototherapy device B1 may be used through a treatment tool channel of an endoscope device inserted into the hollow organ. Even in such a case, the normal tissue other than the abnormal site (lesion) can be prevented from being directly irradiated with light, and the abnormal site can be exclusively irradiated with light.

In addition, in a case where the window B10 is disposed over the entire periphery from the distal end portion B4a toward the proximal end portion B4b of the balloon B4, light can be widely emitted over the entire periphery to a portion limited to a portion extending from the uterine orifice to the vaginal wall. Therefore, in a certain case of gynecological cancer, it is possible to safely treat an abnormal site while suppressing irradiation of light to a normal site of the vaginal wall.

In addition, in a case where the window B10 is disposed only in a portion between the distal end portion B4a and the proximal end portion B4b of the balloon B4, it is possible to irradiate a site limited to the vaginal wall with light, and thus it is possible to safely treat an abnormal site while suppressing irradiation of a normal site of the vaginal wall with light in a certain case of gynecological cancer.

According to the present embodiment, in any configuration, since the light-blocking body B9 is disposed at least at one end portion of the balloon B4 in the longitudinal direction so that the thickness Bt decreases outward in the radial direction of the axial line BO, the light-blocking body B9 is easily deformed as the balloon B4 is deformed outward in the radial direction in which the deformation amount becomes larger when the balloon B4 is deformed from the contracted mode to the expanded mode. As a result, the light-blocking body B9 can be made less likely to be damaged (damage suppressing effect) at the time of expansion of the balloon B4 in which the balloon B4 is deformed from the contracted mode to the expanded mode. In addition, the light emitted from the light emitter B2a can be efficiently blocked by the light-blocking body B9 in which the thickness Bt increases toward the radially inner side where the distance from the light emitter B2a tends to be short (light-blocking efficiency improving effect).

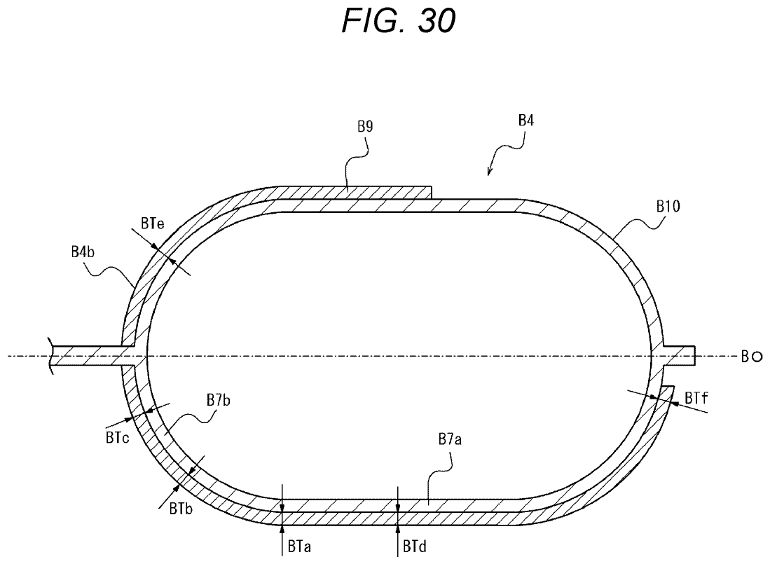

The light-blocking body B9 of the present embodiment may take various forms as long as the light-blocking body B9 is disposed at least at one end portion of the balloon B4 in the longitudinal direction so as to have a different thickness depending on the position. For example, the thickness Bt of the light-blocking body B9 may be set as described in the following [1] to [6]. Here, [1] to [6] can be arbitrarily combined.
[1] The thickness Bt of the light-blocking body B9 may be set such that the thickness BTa (see FIG. 30) of the light-blocking body B9 at a predetermined position of the peripheral wall B7a is smaller than the thickness BTb (see FIG. 30) of the light-blocking body B9 at a predetermined position of the end wall B7b at the proximal end portion B4b of the balloon B4. BTa is preferably in a range of 20 μm to 300 μm, more specifically in a range of 40 μm to 230 μm, and BTb is preferably in a range of 50 μm to 500 μm, more specifically in a range of 150 μm to 450 μm. According to such a configuration, the shape at the time of expansion of the end wall B7b on the proximal side of the balloon B4, which is likely to affect the distribution of the irradiation intensity of light passing through the window B10, can be easily stabilized by the thickness Bt of the light-blocking body 9 at the end wall B7b.
[2] The thickness Bt of the light-blocking body B9 may be set such that, at the proximal end portion B4b of the balloon B4, the thickness BTb of the light-blocking body B9 at a predetermined position of the end wall B7b is smaller than the thickness BTc (see FIG. 30) of the light-blocking body B9 on the radially inner side of the predetermined position of the end wall B7b. In a case where the thickness of the light-blocking body B9 at the boundary between the proximal end portion and the peripheral wall B7a is set to the position of BTb, BTb is preferably in a range of 100 μm to 300 μm, more specifically in a range of 150 μm to 250 μm. In a case where the thickness of the light-blocking body B9 in a portion close the tip end of the proximal end portion is set BTc, BTc is preferably in a range of 150 μm to 500 μm, more specifically in a range of 200 μm to 450 μm. According to such a configuration, the above-described damage suppressing effect and light-blocking efficiency improving effect can be obtained in the end wall B7b at the proximal end portion B4b of the balloon B4.
[3] In a case where the light-blocking body B9 is disposed on the peripheral wall B7a, the thickness Bt of the light-blocking body B9 may be set such that a thickness BTd (see FIG. 30) of the light-blocking body B9 at a predetermined position of the peripheral wall B7a is smaller than the thickness BTa of the light-blocking body B9 at a predetermined position of the peripheral wall B7a proximal of the predetermined position of the peripheral wall B7a where the thickness BTd of the light-blocking body B9 is positioned. In a case where the thickness of the light-blocking body B9 at the distal end of the peripheral wall B7a is at the position of BTa, BTa is preferably in a range of 20 to 300 μm, more specifically in a range of 40 μm to 180 μm. In a case where the thickness of the light-blocking body B9 at the proximal end of the peripheral wall is at the position of BTd, BTd is preferably in a range of 50 μm to 300 μm, more specifically in a range of 100 μm to 230 μm. According to such a configuration, in a certain case of gynecological cancer, the fitting property and the insertability to the shape of the living body (vaginal cavity) can be enhanced.
[4] In a case where the window B10 is disposed to be biased to one side as viewed in the longitudinal direction, the thickness Bt of the light-blocking body B9 may be set such that the thickness BTb or BTa of the light-blocking body B9 at a predetermined position is larger than a thickness BTe (see FIG. 30) at a predetermined position shifted from the predetermined position, where the thickness BTb or BTa of the light-blocking body B9 is positioned, toward the window B10 in the cross-sectional circumferential direction in the proximal end portion B4b of the balloon B4 or other portions. BTe is preferably in a range of 20 μm to 450 μm, more specifically in a range of 40 μm to 430 μm. According to such a configuration, the light emitted from the light emitter B2a can be efficiently blocked.
[5] The thickness Bt of the light-blocking body B9 may be set such that a thickness BTf (see FIG. 30) at an edge portion of the light-blocking body B9 forming a boundary with the window B10 is smaller than a thickness (for example, the thicknesses BTa, BTb, and the like described above) of the light-blocking body B9 at other positions. BTf is preferably in a range of 1 μm to 200 μm, more specifically in a range of 10 μm to 150 μm. According to such a configuration, it is possible to suppress damage to the tissues surrounding the living body and the light-blocking body B9 itself when the balloon B4 moves in the living body.
[6] The thickness Bt of the light-blocking body B9 may be set to gradually change as the position changes. According to such a configuration, the above effect can be enhanced. Note that the present disclosure is not limited thereto, and the thickness Bt of the light-blocking body B9 may be set to change in a stepped shape when the position changes.

The second aspect of the present disclosure is not limited to the above-described embodiment and may be modified in various manners without departing from the gist of the present disclosure.

Therefore, the phototherapy device B1 according to the embodiment of the second aspect described above can be variously changed and includes the balloon B4 that forms the internal space B3 in which the light emitter B2a is disposed and defines the longitudinal direction and the axial line BO extending in the longitudinal direction, and the lumen B6 serving as a movement path of the fluid that deforms the balloon B4 from the contracted mode to the expanded mode. The light emitter B2a is disposed on the axial line BO in the internal space B3. The balloon B4 includes the light-blocking body B9 that blocks at least a part of the light emitted from the light emitter B2a from being transmitted toward the outside of the internal space B3, and the window B10 that transmits the light toward the outside of the internal space B3 due to absence of the light-blocking body B9. The light-blocking body B9 is disposed at least at one end portion of the balloon B4 in the longitudinal direction so as to have a different thickness depending on the position.

For example, the light emitter B2a is not limited to the configuration capable of emitting light toward the entire region in the circumferential direction of the axial center, and may be configured to emit light toward only a partial region in the circumferential direction of the axial center, for example. The phototherapy device B1 is not limited to the configuration in which the light emitter B2a is configured to be introduced into the internal space B3 of the hollow body by movement in the optical lumen B5, and for example, the light emitter B2a may be incorporated in the internal space B3 of the hollow body.

Note that the phototherapy device B1 according to the above-described embodiment is preferably a phototherapy device B1 in which the fluid is introduced into the internal space B3 through the one end portion of the balloon B4 in the lumen B6 serving as a movement path of the fluid.

The phototherapy device B1 according to the above-described embodiment is preferably a phototherapy device B1 in which the thickness Bt of the light-blocking body B9 is set such that the thickness BTa of the light-blocking body B9 at a predetermined position of the peripheral wall B7a is smaller than the thickness BTb of the light-blocking body B9 at a predetermined position of the end wall B7b at the proximal end portion B4b of the balloon B4.

The phototherapy device B1 according to the above-described embodiment is preferably a phototherapy device B1 in which the thickness Bt of the light-blocking body B9 is set such that the thickness BTb of the light-blocking body B9 at a predetermined position of the end wall B7b is smaller than the thickness BTc of the light-blocking body B9 on the radially inner side of the predetermined position of the end wall B7b in the proximal end portion B4b of the balloon B4.

The phototherapy device B1 according to the above-described embodiment is preferably a phototherapy device B1 in which the thickness Bt of the light-blocking body B9 is set such that, in a case where the light-blocking body B9 is disposed on the peripheral wall B7a of the balloon B4, the thickness BTd of the light-blocking body B9 at a predetermined position of the peripheral wall B7a is smaller than the thickness BTa of the light-blocking body B9 at a predetermined position of the peripheral wall B7a proximal of the predetermined position of the peripheral wall B7a where the thickness BTd of the light-blocking body B9 is positioned.

The phototherapy device B1 according to the above-described embodiment is preferably a phototherapy device B1 in which the thickness Bt of the light-blocking body B9 is set so that, in a case where the window B10 is disposed to be biased to one side as viewed in the longitudinal direction, the thickness BTb of the light-blocking body B9 at a predetermined position is larger than the thickness BTe at a predetermined position shifted from the predetermined position, where the thickness BTb of the light-blocking body B9 is positioned, toward the window B10 in the circumferential direction.

The phototherapy device B1 according to the above-described embodiment is preferably a phototherapy device B1 in which the thickness Bt of the light-blocking body B9 is set such that the thickness BTf at the edge portion of the light-blocking body B9 forming the boundary with the window B10 is smaller than the thickness of the light-blocking body B9 at other positions.

The phototherapy device B1 according to the above-described embodiment is preferably a phototherapy device B1 in which the thickness Bt of the light-blocking body B9 is set to gradually change as the position changes.

The phototherapy device B1 according to the above-described embodiment is preferably manufactured by a method for forming the light-blocking body B9 by a dipping method.

Next, an embodiment of a third aspect of the present disclosure will be described. A phototherapy device C1 according to an embodiment of the third aspect of the present disclosure includes an enclosure (hollow body C4) that forms an internal region (internal space C3) in which a light emitter C2a is disposed and defines a longitudinal direction and an axial line (axial center CP) extending in the longitudinal direction. The enclosure includes a light-blocking body (reflector C9) that blocks at least part of light emitted from the light emitter C2a from being transmitted toward the outside of the internal region, and a window C10 that transmits light toward the outside of the internal region due to absence of the light-blocking body. The window C10 may extend from one end portion of the window C10 located at one end portion of the enclosure in the longitudinal direction toward the other end portion of the enclosure, and one end portion C10 in the longitudinal direction may be disposed over a limited angular range not extending over the entire periphery in the circumferential direction of the axial line. The light emitter C2a is disposed on the axial line of the enclosure. The light emitter C2a may be disposed so as not to overlap the window C10 as viewed in the radial direction.

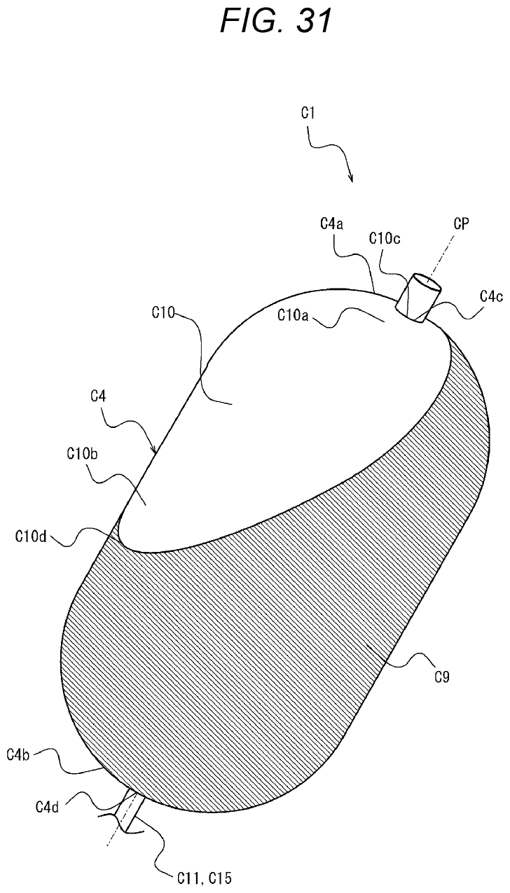

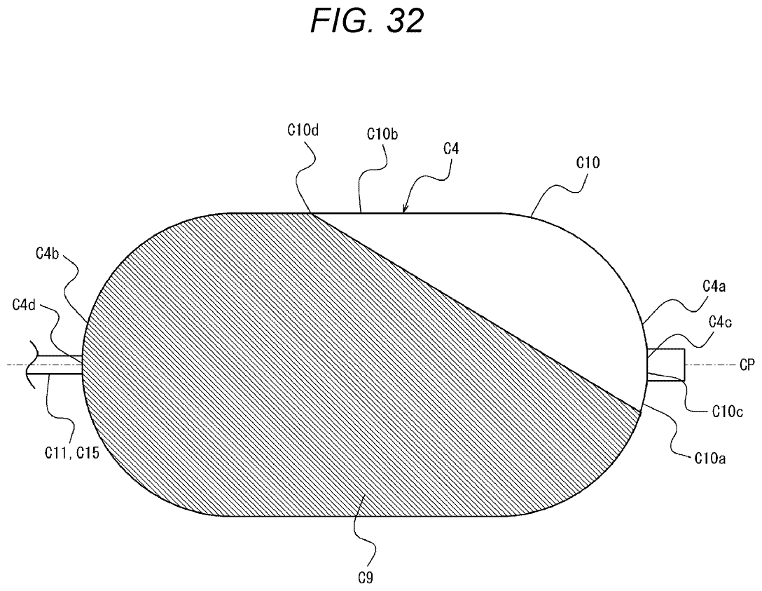

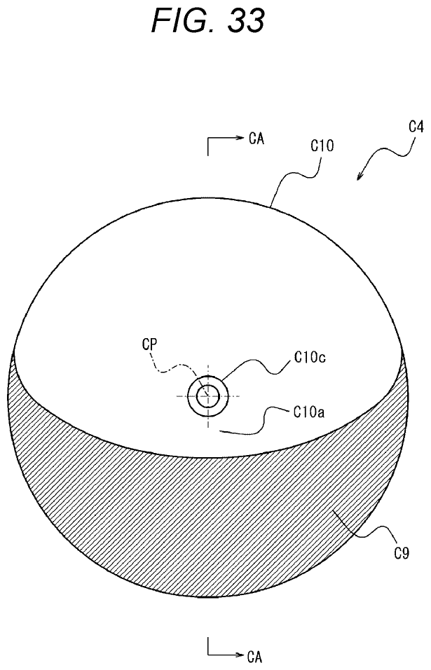

The phototherapy device C1 according to an embodiment of the third aspect of the present disclosure illustrated in FIGS. 31 to 36 is used to irradiate abnormal tissue with therapeutic light in a phototherapeutic treatment such as photoimmunotherapy (PIT) or photodynamic therapy (PDT). The light can be, for example, laser light.

The phototherapy device C1 includes a light emitting device C2 having a light emitter C2a defining a longitudinal direction and a central axial line CO extending in the longitudinal direction, a hollow body C4 forming an internal space C3 in which the light emitter C2a is disposed, and a lumen C5 (hereinafter, also referred to as an optical lumen C5) serving as a movement path of the light emitter C2a.

The light emitter C2a is elongated in a predetermined direction, and defines a longitudinal direction as the predetermined direction. Note that a direction along a straight line perpendicular to the central axial line CO is also referred to as a radial direction or simply a radial direction of the central axial line CO, and a direction around the central axial line CO is also referred to as a circumferential direction or simply a circumferential direction of the central axial line CO.

The hollow body C4 is elongated along the axial center CP, and has a tubular shape in which both end portions in a direction along the axial center CP (hereinafter, also referred to as an axial direction) are closed. The light emitter C2a is disposed on the axial center CP of the hollow body C4 in the internal space C3, and preferably a portion over the entire length in the axial direction of the light emitter C2a is disposed on the axial center CP, and more preferably the light emitter C2a is disposed so as to coincide with the axial center CP.

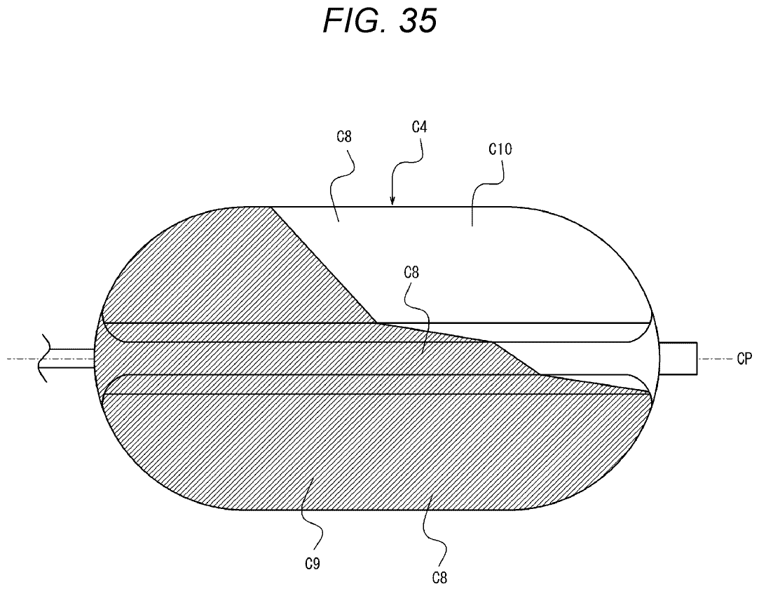

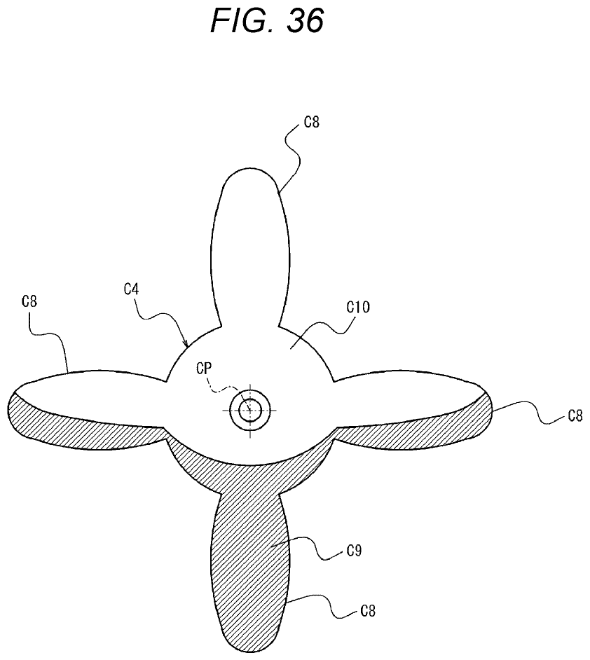

The hollow body C4 is a balloon that is configured to be deformable from the contracted mode illustrated in FIGS. 35 and 36 to the developed mode illustrated in FIGS. 31 to 34, and the phototherapy device C1 further includes a lumen C6 (hereinafter, the fluid lumen is also referred to as a fluid lumen C6) serving as a movement path of fluid that deforms the balloon from the contracted mode to the developed mode. The hollow body C4 can be deformed from the contracted mode to the developed mode by the pressure of the fluid moving in the fluid lumen C6 and entering the internal space C3. The hollow body C4 (balloon) may be configured to be deformable from the contracted mode to the developed mode by means other than fluid.

In the developed mode, the hollow body C4 has a cylindrical shape in which both end portions in the axial direction are closed around the axial center CP. More specifically, the hollow body C4 includes a peripheral wall C7a having a cylindrical shape in the developed mode, one end wall C7b having a convex curved shape continuous with one end portion of the peripheral wall C7a and protruding outward in the axial direction in the developed mode, and another end wall C7b having a convex curved shape continuous with the other end portion of the peripheral wall C7a and protruding outward in the axial direction in the developed mode.

In the hollow body C4, both or either one of the end walls C7b may have a convex shape other than a convex curved shape in the developed mode. The hollow body C4 may have a configuration in which the peripheral wall C7a has a tubular shape other than a cylindrical shape, such as an elliptical cylindrical shape in the developed mode. That is, the hollow body C4 may have a tubular shape other than a cylindrical shape in which both end portions in the axial direction are closed around the axial center CP in the developed mode.

The contracted mode is a mode in which the hollow body C4 is folded into a predetermined shape so that the internal space C3 is reduced from the developed mode. The predetermined shape may be a shape in which a plurality of pleats C8 are arranged in the circumferential direction, and may be, for example, a shape in which four pleats C8 are formed as illustrated in FIGS. 35 and 36.

The configuration of the hollow body C4 is not limited to the balloon that is configured to be deformable from the contracted mode to the developed mode, and may be, for example, a configuration that always corresponds to the above-described developed mode.

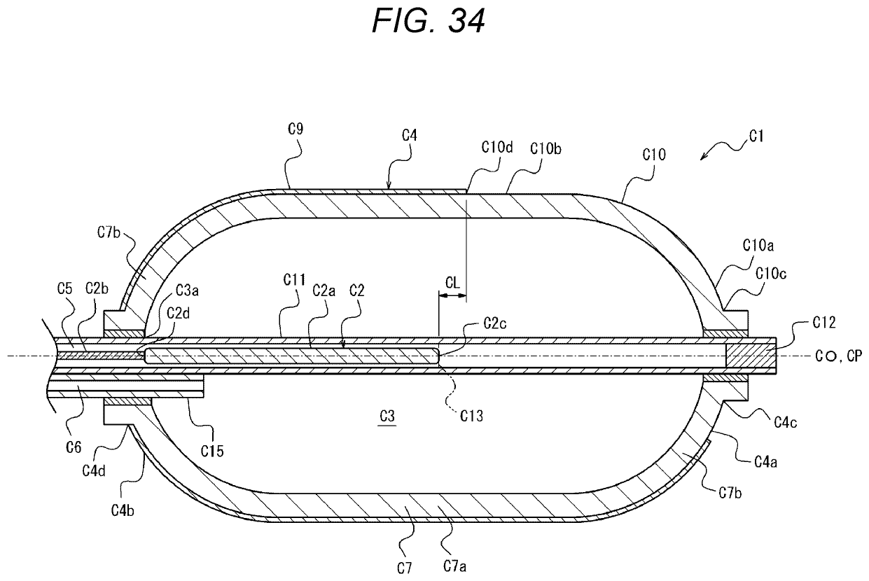

As illustrated in FIG. 34, the light emitting device C2 further includes a light source and an elongated light guide unit C2b that transmits the light emitted from the light source to the light emitter C2a. The light guide unit C2b is configured by, for example, a light guide such as an optical fiber, and is continuous with the light source at one end portion and is continuous with the light emitter C2a at the other end portion. In addition, the light guide unit C2b may have an optical connector that is connected to the light guide and is configured to be detachable from the light source.

The light emitter C2a extends in an elongated shape along the central axial line CO, and is configured to be capable of emitting light transmitted from the light source via the light guide unit C2b in the radial direction from a portion over a predetermined width in the longitudinal direction. More specifically, the light emitter C2a can emit light toward the entire region in the circumferential direction.

The hollow body C4 includes a light-blocking body C9 that blocks at least part of light emitted from the light emitter C2a from being transmitted toward the outside of the internal space C3, and a window C10 that transmits light toward the outside of the internal space C3 due to absence of the light-blocking body C9. More specifically, the hollow body C4 includes a main body C7 having a peripheral wall C7a and two end walls C7b, and a light-blocking body C9 covering a part of an outer surface of the main body C7, and the window C10 is configured by a portion of the main body C7 that is not covered with the light-blocking body C9. The main body C7 is formed of a light transmitting material, and the light-blocking body C9 is formed of a light blocking material. The light-blocking body C9 may be configured as a reflector that at least partially reflects the light emitted from the light emitter C2a toward the internal region (internal space C3). In this case, the light-blocking body C9 is formed of a light reflective material. The main body C7 can be made of, for example, a resin material, and is preferably made of polyethylene terephthalate, polyurethane, nylon, or the like. According to the light-blocking body C9 as a reflector, the light emitted from the light emitter C2a can be collected and efficiently emitted to a limited range through the window C10.

The method for forming the light-blocking body C9 is not particularly limited, and the light-blocking body C9 can be formed by, for example, coating to the main body C7. The coating method is not particularly limited, and for example, a dipping method, a spray coating method, a roll coating method, a screen printing method, or the like can be used. The light-blocking body C9 is not limited to cover a part of the outer surface of the main body C7, but may cover a part of the inner surface of the main body C7, for example.

The window C10 extends from one end portion (hereinafter, also referred to as a distal end portion C10a) of the window C10 located at one end portion (hereinafter, also referred to as a distal end portion C4a) of the hollow body C4 in the axial direction toward the other end portion (hereinafter, also referred to as a proximal end portion C4b) of the hollow body C4. In addition, a tip end C10c of the distal end portion C10a of the window C10 is located at a tip end C4c of the distal end portion C4a of the hollow body C4. That is, the window C10 is disposed in a region including the center of the hollow body C4 as viewed in the axial direction.

The other end portion (hereinafter, also referred to as a proximal end portion C10b) of the window C10 in the axial direction is disposed over a limited angular range not over the entire periphery in the circumferential direction of the axial center CP. That is, the proximal end portion C10b of the window C10 is disposed only in a part of the axial center CP in the circumferential direction. An angular range in which the window C10 is disposed is set so as to be gradually narrowed from the distal end portion C10a toward the proximal end portion C10b of the window C10. A tip end C10d of the proximal end portion C10b of the window C10 is located on the peripheral wall C7a. More specifically, the tip end C10d of the proximal end portion C10b of the window C10 is located on the proximal end side with respect to the center of the hollow body C4 in the axial direction. The angular range in the circumferential direction of the axial center CP at the tip end C10c is preferably a range of 180 degrees to 360 degrees, more specifically 360 degrees. The angular range in the circumferential direction of the axial center CP in the vicinity of the tip end C10c of the distal end portion C10a is preferably a range of 180 degrees to 360 degrees, more specifically 360 degrees. The angular range in the circumferential direction of the axial center CP in the portion located on the end wall C7b on the distal end side forming a convex curved shape in the range from the vicinity of the distal end portion C10a to the tip end C10d is a range of 45 degrees to 360 degrees, more specifically a range of 60 degrees to 360 degrees, and preferably a range of 90 degrees to 360 degrees. The angular range in the circumferential direction of the axial center CP at the tip end C10d is a range of 45 degrees to 180 degrees, more specifically a range of 60 degrees to 120 degrees, and preferably 90 degrees.

The light emitter C2a is introduced into the internal space C3 through a tip end C4d of the proximal end portion C4b of the hollow body C4 within the optical lumen C5. The optical lumen C5 is formed by a tube C11 (hereinafter, also referred to as an optical tube C11) arranged coaxially with the axial center CP, wherein the optical tube C11 passes through a proximal end portion C4b and a distal end portion C4a of the hollow body C4. More specifically, the optical tube C11 is bonded to the end wall C7b at the proximal end portion C4b of the hollow body C4, and bonded to the end wall C7b at the distal end portion C4a of the hollow body C4. The optical tube C11 is closed by a closing member C12 at the distal end portion C4a of the hollow body C4. The optical tube C11 has optical transparency.

In the present embodiment, the light emitter C2a is disposed at a predetermined position where the light emitter C2a does not overlap the window C10 as viewed in the radial direction in the internal space C3. That is, when the light emitter C2a is disposed at the predetermined position, a distal end C2c (that is, the tip end on the side from the proximal end portion C4b to the distal end portion C4a of the hollow body C4 in the axial direction) of the light emitter C2a is retracted toward the proximal end portion C4b of the hollow body C4 by a distance CL (where L≥0) from the tip end C10d of the proximal end portion C10b of the window C10 in the longitudinal direction.

In a case where the window C10 extends from the distal end portion C10a of the window C10 located at the distal end portion C4a of the hollow body C4 toward the proximal end portion C4b of the hollow body C4, and the proximal end portion C10b of the window C10 is disposed over a limited angular range not extending over the entire periphery in the circumferential direction of the axial center CP, it is possible to suppress uneven irradiation of light to the outside of the hollow body C4 through the window C10 by arranging the light emitter C2a at a predetermined position so as not to overlap the window C10 as viewed in the radial direction.

In order to easily arrange the light emitter C2a at a predetermined position, the optical tube C11 includes a marker C13 such as a contrast marker having radiopacity serving as a mark for arranging the light emitter C2a at a predetermined position. In the example shown, the light emitter C2a is arranged such that the distal end C2c of the light emitter C2a coincides with the marker C13.

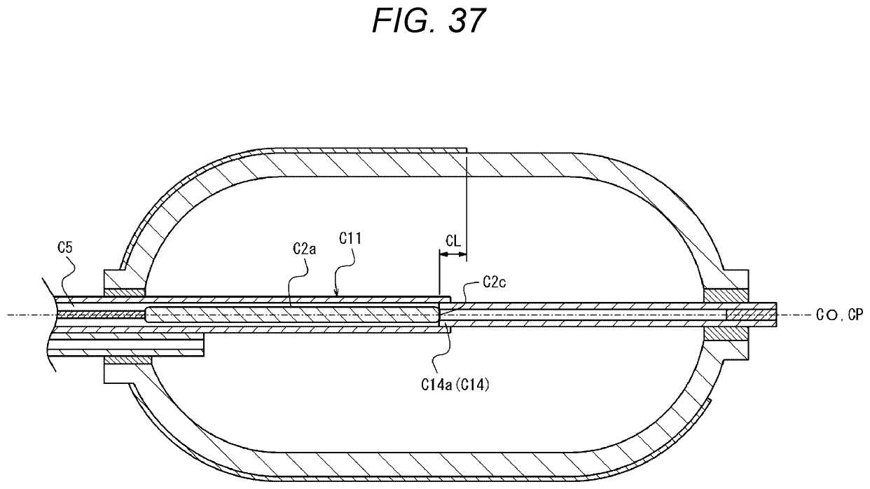

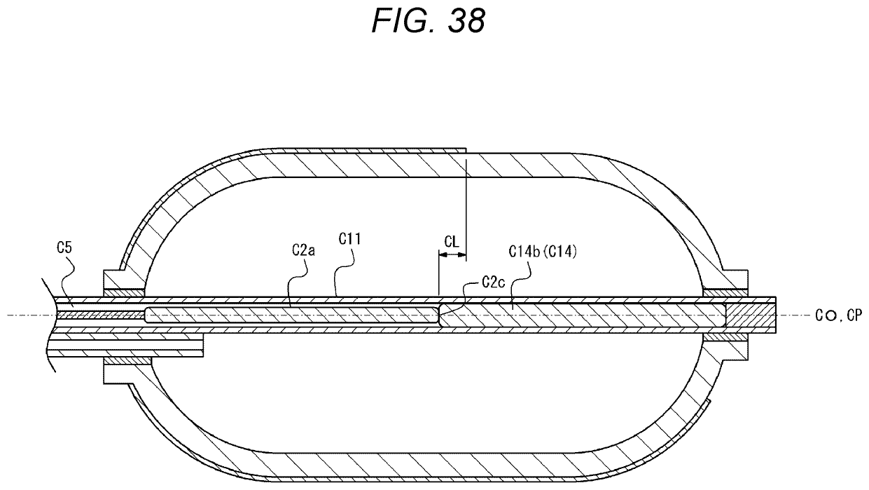

Alternatively, as illustrated in FIGS. 37 and 38, the optical tube C11 may have a stopper C14 that restricts the movement of the light emitter C2a in the introduction direction in the optical lumen C5 by hitting the light emitter C2a (for example, the distal end C2c) at a predetermined position. The stopper C14 may be constituted by a diameter-reduced portion C14a disposed in the optical tube C11 as illustrated in FIG. 37, or may be constituted by a member C14b disposed in the optical tube C11 as illustrated in FIG. 38.

In the present embodiment, when the light emitter C2a is disposed at the predetermined position, the proximal end C2d (that is, the tip end on the side from the distal end portion C4a toward the proximal end portion C4b of the hollow body C4 in the axial direction) of the light emitter C2a is located at a proximal end C3a of the internal space C3 in the longitudinal direction. According to such a configuration, it is possible to efficiently emit light.

Fluid is introduced into the internal space C3 through the proximal end portion C4b of the hollow body C4 within the fluid lumen C6 to deform the hollow body C4 from the contracted mode to the developed mode. The fluid lumen C6 is formed by a tube C15 (hereinafter, also referred to as a fluid tube C15) disposed adjacent to and in parallel with the optical tube C11, and the fluid tube C15 passes through the proximal end portion C4b of the hollow body C4. More specifically, the fluid tube C15 is adhered to the end wall C7b at the proximal end portion C4b of the hollow body C4. The fluid tube C15 opens in the internal space C3.

The fluid lumen C6 is not limited to the configuration disposed in parallel with the optical lumen C5, and for example, may be disposed on the radial outside of the optical lumen C5 coaxially with the optical lumen C5, or may be configured with a common lumen that is configured to be used as both the fluid lumen C6 and the optical lumen C5.

According to the phototherapy device C1 of the present embodiment, for example, in the case of treating gynecological cancer, the hollow body C4 is deformed from the contracted mode to the developed mode in front of the uterine orifice, and light can be widely emitted from the distal end portion C10a of the window C10 to the vicinity of the uterine orifice, and at the same time, light can be limitedly emitted to the vaginal wall from the remaining portion of the window C10 in the circumferential direction. Therefore, it is possible to treat the abnormal site while suppressing the irradiation of the normal site of the vaginal wall with light. At that time, the region extending from the vicinity of the uterine orifice to the abnormal site of the vaginal wall can be irradiated with light while suppressing uneven irradiation. According to such a phototherapy device C1, it is possible to safely treat a certain case of gynecological cancer. Note that the phototherapy device C1 may be applied to cancer other than gynecological cancer (for example, esophageal cancer or gastric cancer). For example, in a case where the phototherapy device C1 is applied to a hollow organ such as a digestive tract (esophagus, stomach, small intestine, large intestine, etc.), a urinary tract, and a blood vessel, the phototherapy device C1 may be used through a treatment tool channel of an endoscope device inserted into the hollow organ. Even in such a case, the normal tissue other than the abnormal site (lesion) can be prevented from being directly irradiated with light, and the abnormal site can be exclusively irradiated with light.

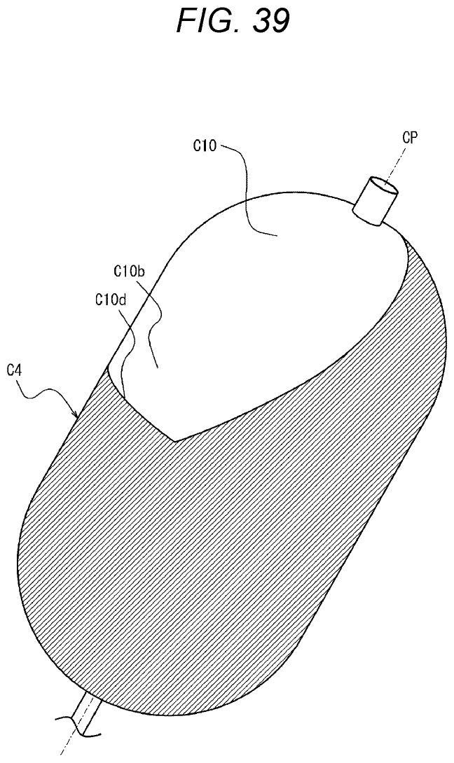

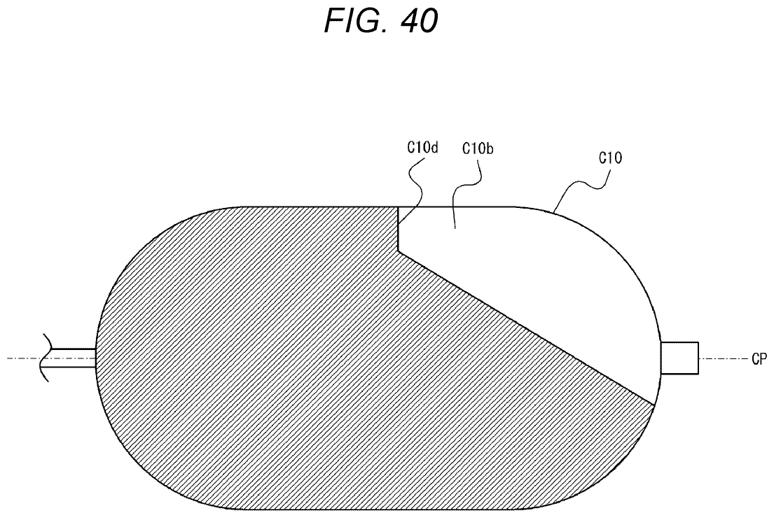

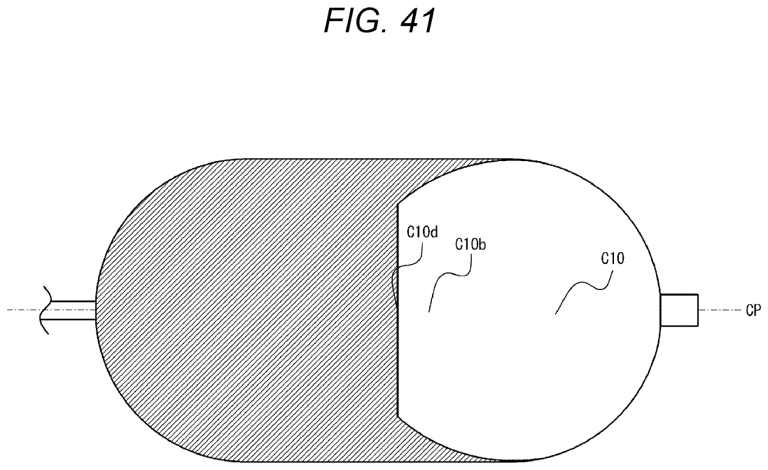

As in the second embodiment illustrated in FIGS. 39 to 41, the window C10 may have a configuration in which the tip end C10d of the proximal end portion C10b of the window C10 extends along the circumferential direction. Also in this case, the light emitter C2a is disposed at the predetermined position so as not to overlap the window C10 as viewed in the radial direction. According to such a configuration, light can be emitted with a constant width in the circumferential direction at the tip end C10d of the proximal end portion C10b of the window C10, and at that time, the region extending from the vicinity of the uterine orifice to the abnormal site of the vaginal wall can be irradiated with light while suppressing uneven irradiation, so that it is possible to efficiently irradiate light in a certain case of gynecological cancer.

The third aspect of the present disclosure is not limited to the above-described embodiment and may be modified in various manners without departing from the gist of the present disclosure.

Therefore, the phototherapy device C1 according to the embodiment of the third aspect described above can be variously changed and includes the light emitter C2a that defines the longitudinal direction and the central axial line CO extending in the longitudinal direction and emits light in the radial direction of the central axial line CO, and the hollow body C4 that forms the internal space C3 in which the light emitter C2a is disposed. The light emitter C2a is disposed on the axial center CP of the hollow body C4. The hollow body C4 includes the light-blocking body C9 that blocks at least a part of light from being transmitted toward the outside of the internal space C3, and the window C10 that transmits light toward the outside of the internal space C3 due to absence of the light-blocking body C9. The window C10 extends from one end portion of the window C10 located on one end portion side of the hollow body C4 in the axial direction along the axial center CP toward the other end portion of the hollow body C4, and the other end portion of the window C10 in the axial direction is disposed over a limited angular range not extending over the entire periphery in the circumferential direction of the axial center CP. The light emitter C2a is disposed so as not to overlap the window C10 as viewed in the radial direction

For example, the light emitter C2a is not limited to the configuration capable of emitting light toward the entire region in the circumferential direction, and may be configured to emit light toward only a partial region in the circumferential direction, for example. The phototherapy device C1 is not limited to the configuration in which the proximal end C2d of the light emitter C2a is located at the proximal end C3a of the internal space C3 in the longitudinal direction when the light emitter C2a is disposed at the predetermined position. The phototherapy device C1 is not limited to the configuration in which the light emitter C2a is configured to be introduced into the predetermined position in the internal space C3 of the hollow body C4 by movement in the optical lumen C5, and for example, the light emitter C2a may be built in the predetermined position in the internal space C3 of the hollow body C4. The tip end C10d of the proximal end portion C10b of the window C10 may be located on the distal end side with respect to the center of the hollow body C4 in the axial direction. The window C10 is not limited to the configuration in which the distal end portion C10a of the window C10 is located at the distal end portion C4a of the hollow body C4, and the distal end portion C10a of the window C10 may be located on the proximal side of the distal end portion C4a of the hollow body C4.

Note that the phototherapy device C1 according to the above-described embodiment is preferably a phototherapy device C1 in which the one end portion of the window C10 is located at the one end portion of the hollow body C4.

The phototherapy device C1 according to the above-described embodiment is preferably a phototherapy device C1 having a lumen C5 serving as a movement path of the light emitter C2a.

The phototherapy device C1 according to the above-described embodiment is preferably a phototherapy device C1 in which the light emitter C2a is introduced into an internal region through the other end portion of the hollow body C4 in the lumen C5 serving as a movement path of the light emitter C2a.

The phototherapy device C1 according to the above-described embodiment is preferably a phototherapy device C1 including the tube C11 forming the lumen C5 serving as a movement path of the light emitter C2a, and the tube C11 including the marker C13 indicating the position where the light emitter C2a is to be disposed.

The phototherapy device C1 according to the above-described embodiment is preferably a phototherapy device C1 including the stopper C14 that restricts the movement of the light emitter C2a in the introduction direction in the lumen C5, which is the movement path of the light emitter C2a, at a position where the light emitter C2a is to be disposed.

The phototherapy device C1 according to the above-described embodiment is preferably a phototherapy device C1 in which the tip end C10c of the one end portion of the window C10 is located at the tip end C4c of the one end portion of the hollow body C4.

The phototherapy device C1 according to the above-described embodiment is preferably a phototherapy device C1 in which the hollow body C4 is a balloon that is configured to be deformable from the contracted mode to the developed mode.

The phototherapy device C1 according to the above-described embodiment is preferably a phototherapy device C1 having the lumen C6 serving as a movement path of the fluid that deforms the hollow body C4 from the contracted mode to the developed mode.

The phototherapy device C1 according to the above-described embodiment is preferably a phototherapy device C1 in which fluid is introduced into an internal region through the other end portion of the hollow body C4 in the lumen C6 serving as a fluid movement path.

The detailed description above describes embodiments of a phototherapy device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A phototherapy device comprising:
an enclosure that encloses an internal region where a light emitter is disposed and defines a longitudinal direction and an axial line extending in the longitudinal direction, wherein
the enclosure includes a light-blocking body that blocks at least a part of light emitted from the light emitter from being transmitted toward an outside of the internal region, and a window that transmits the light toward an outside of the internal region due to absence of the light-blocking body,
the window extends from one end portion of the window located at one end portion of the enclosure in the longitudinal direction toward an other end portion of the enclosure, and
wherein an other end portion of the window in the longitudinal direction is disposed over a limited angular range not extending over an entire periphery in a circumferential direction of the axial line.

2. The phototherapy device according to claim 1, wherein a tip end of the one end portion of the window is located at a tip end of the one end portion of the enclosure.

3. The phototherapy device according to claim 1, further comprising:
a lumen serving as a movement path of the light emitter.

4. The phototherapy device according to claim 3, wherein the light emitter is introduced into the internal region through the other end portion of the enclosure in the lumen serving as a movement path of the light emitter.

5. The phototherapy device according to claim 1, wherein the other end portion of the window is defined by a straight line perpendicular to the longitudinal direction.

6. The phototherapy device according to claim 1, wherein the enclosure includes an absorber that at least partially absorbs the light emitted from the light emitter, transmitted through the enclosure, and then incident on the enclosure from outside.

7. The phototherapy device according to claim 1, wherein the other end portion of the enclosure has a labeling body indicating an orientation of the window in the circumferential direction.

8. The phototherapy device according to claim 1, comprising:
a lumen serving as a movement path of fluid that deforms the enclosure from a contracted mode to an expanded mode, wherein the light emitter is disposed on the axial line in the internal region, and
the light-blocking body is disposed at least at one end portion of the enclosure in the longitudinal direction so as to have a different thickness depending on a position.

9. The phototherapy device according to claim 1, wherein
the light emitter is disposed on an axial center of the enclosure, and
the light emitter is disposed so as not to overlap the window as viewed in a radial direction of the axial line.

10. The phototherapy device according to claim 9, wherein the one end portion of the window is located at the one end portion of the enclosure.

11. The phototherapy device according to claim 9, further comprising:
a lumen serving as a movement path of the light emitter.

12. The phototherapy device according to claim 9, wherein a tip end of the one end portion of the window is located at a tip end of the one end portion of the enclosure.

13. A phototherapy method using the phototherapy device, the phototherapy device an enclosure that encloses an internal region where a light emitter is disposed and defines a longitudinal direction and an axial line extending in the longitudinal direction, wherein the enclosure includes a light-blocking body that blocks at least a part of light emitted from the light emitter from being transmitted toward an outside of the internal region, and a window that transmits the light toward an outside of the internal region due to absence of the light-blocking body, the window extends from one end portion of the window located at one end portion of the enclosure in the longitudinal direction toward an other end portion of the enclosure, and an other end portion of the window in the longitudinal direction is disposed over a limited angular range not extending over an entire periphery in a circumferential direction of the axial line, the method comprising:
inserting the enclosure into a living body;
rotating the enclosure in the circumferential direction in accordance with a position of a tumor;
emitting the light from the enclosure through the window; and
removing the enclosure from the living body.

14. The phototherapy method according to claim 13, comprising:
visually recognizing a uterine orifice and an affected area with a colposcope or another visual recognition instrument inserted into a vagina; and
removing the visual recognition instrument from an inside of the vagina, wherein
the visual recognizing is performed before the inserting, the rotating, the emitting, the removing, and the expanding, and
the removing of the visual recognition instrument is performed after the inserting and before the emitting.

15. The phototherapy method according to claim 13, further comprising:
confirming that the enclosure has been abutted against a deepest part of a vagina by the inserting.

16. The phototherapy method according to claim 13, further comprising:
irradiating an entire inside of the living body with the light having output power smaller than output power in the emitting, wherein
the irradiating is performed after the emitting.

17. The phototherapy method according to claim 13, further comprising:
re-emitting the light by changing a position of an emission range in the living body after the emitting.

18. The phototherapy method according to claim 17, further comprising:
changing a position of the enclosure by rotating the enclosure in the circumferential direction or moving the enclosure in the longitudinal direction, wherein
the changing is performed after the emitting and before the re-emitting.

19. The phototherapy method according to claim 13, wherein
a first phototherapy device is used as the phototherapy device, the first phototherapy device including a first enclosure that encloses a first internal region in which a first light emitter is disposed and defines a first longitudinal direction and a first axial line extending in the first longitudinal direction, the first enclosure including a first light-blocking body that blocks at least part of first light emitted from the first light emitter from being transmitted toward an outside of the first internal region and a first window that transmits the first light toward an outside of the first internal region due to absence of the first light-blocking body, the first window extending from one end portion of the first window located at one end portion of the first enclosure in the first longitudinal direction toward an other end portion of the first enclosure, and an other end portion of the first window in the first longitudinal direction being disposed over a limited angular range not extending over an entire periphery in a circumferential direction of the first axial line,
a second phototherapy device is used, the second phototherapy device including a second enclosure that encloses a second internal region in which a second light emitter is disposed and defines a second longitudinal direction and a second axial line extending in the second longitudinal direction, the second enclosure including a second light-blocking body that blocks at least part of second light emitted from the second light emitter from being transmitted toward an outside of the second internal region, and a second window that transmits the second light toward an outside of the second internal region due to absence of the second light-blocking body, and the second window being disposed in a portion other than both end portions of the second enclosure in the second longitudinal direction, and the phototherapy method further comprises:
inserting, as the inserting using the first phototherapy device, the first enclosure into the living body;
rotating, as the rotating using the first phototherapy device, the first enclosure in a first circumferential direction in accordance with a position of the tumor;
emitting, as the emitting using the first phototherapy device, the first light from the first enclosure through the first window;
removing, as the removing using the first phototherapy device, the first enclosure from the living body;
inserting, using the second phototherapy device, the second enclosure into the living body;
rotating, using the second phototherapy device, the second enclosure in the second circumferential direction in accordance with a position of the tumor;
emitting, using the second phototherapy device, the second light from the second enclosure through the second window; and
removing, using the second phototherapy device, the second enclosure from the living body.

20. A phototherapy device comprising:
a balloon that forms an internal space in which a light emitter is disposed and defines a longitudinal direction and an axial line extending in the longitudinal direction; and
a lumen serving as a movement path of fluid that deforms the balloon from a contracted mode to an expanded mode, wherein
the light emitter is disposed on the axial line in the internal space,
the balloon includes a light-blocking body that blocks at least a part of light emitted from the light emitter from being transmitted toward an outside of the internal space, and a window that transmits the light toward an outside of the internal space due to absence of the light-blocking body, and
the light-blocking body is disposed at least at one end portion of the balloon in the longitudinal direction so as to have a different thickness depending on a position.
